(19) United States
(12) Patent Application Publication
(10) Pub. No.: US 2010/0137263 A1
(43) Pub. Date: Jun. 3, 2010

(54) ASSAY FOR THE DETECTION OF BIOMARKERS ASSOCIATED WITH PREGNANCY RELATED CONDITIONS

(75) Inventor: Roger Smith, Callaghan (AU)

Correspondence Address: MERCHANT & GOULD PC, P.O. BOX 2903, MINNEAPOLIS, MN, 55402-0903, US

(73) Assignee: NEWCASTLE INNOVATION LIMITED, Callaghan (AU)

(21) Appl. No.: 12/446,363

(22) PCT Filed: Oct. 19, 2007
(86) PCT No.: PCT/AU2007/001598

§ 371 Date: Oct. 23, 2009

(30) Foreign Application Priority Data
Oct. 20, 2006 (EP) 06122703.9
Oct. 20, 2006 (EP) 06122704.7
Oct. 20, 2006 (EP) 06122705.4

(51) Int. Cl. A61K 31/56; C12Q 1/00; G01N 33/00; A61P 15/06

(52) U.S. Cl. 514/169; 435/4; 436/86

(57) ABSTRACT

The invention relates to screening methods for determination of the risk of preterm delivery and/or pregnancy associated conditions. The methods involve detection of the level of one or more biomarkers in a biological sample from the patient. In particular, in embodiments of the invention there are provided methods for determining the risk of pre-eclampsia and other hypertensive disorders, and intrauterine growth retardation (IGUR).

### FIELD OF THE INVENTION

This invention relates to screening methods for determining the risk of preterm delivery and pregnancy associated conditions involving the detection of levels of one or more biomarkers in biological samples from pregnant women.

### BACKGROUND OF THE INVENTION

Every year approximately 4.5 million premature babies are born worldwide, and, despite considerable advances in neonatal care, their mortality rate remains high. Moreover, survivors are at risk for long-term handicap, including developmental delay cerebral palsy, blindness, deafness, and chronic lung disease. In particular, preterm neonates account for more than half, and maybe as much as three-quarters of the morbidity and mortality of newborns without congenital anomalies.

Determination of impending preterm births is critical for increasing neonatal survival of preterm infants. Its limited success has been attributed, in part, to the fact that premature parturition is a syndrome caused by multiple pathological processes such as infection, vascular disease, uterine over-distension, and chronic stress.

Although tocolytic agents which can delay delivery were introduced 20 to 30 years ago, there has been only a minor decrease in the incidence of preterm delivery. It has been postulated that the failure to observe a larger reduction in the incidence of preterm births is due to errors in the diagnosis of preterm labour and to the patients conditions being too advanced for tocolytic agents to successfully delay the birth.

Specifically, the diagnosis and the therapeutic treatment of a threat of preterm delivery remain unsolved clinical problems. Rapid identification of patients at risk is difficult. Conventional methods of prognosis, which are based on the analysis of obstetric history, of demographic factors and of premonitory symptoms, are neither objective, nor sensitive, nor specific.

Many studies have been carried out in order to search for biochemical markers correlated with a threat of preterm delivery, which would allow reliable and rapid diagnosis able to be used to monitor patients at risk.

WO 2006/086731 discloses a method for assessing the risk of preterm delivery wherein the level of methyladenine, heptanedioic acid and N-acetyl glutamine is determined in a biological sample, preferably amniotic fluid, obtained from a pregnant subject. US 2006/0127962 discloses a diagnostic assay for determining a biomarker indicative of intra amniotic inflammation in a sample of amniotic fluid wherein the biomarker is a calgranulin. Amniotic samples are obtained by amniocentesis, a method which in itself increases the risk of foetal loss by approximately 1%.

WO 01/88545 concerns a method for forecasting impending preterm delivery by detecting the presence or absence of IL-6 in a cervicovaginal secretion sample. WO 93/24836 relates to a method wherein the levels of defensins are determined in a cervicovaginal secretion sample from a pregnant patient in order to establish an increased risk of preterm delivery. Cervicovaginal secretion samples are not easily obtained and in many cultures vaginal fluid sampling would not readily be accepted as part of a routine screening assay.

Foetal fibronectin in vaginal fluid is also often used as a biomarker for imminent delivery. Such markers may predict delivery within a week or two, however, they are not indicative of preterm delivery for months in advance.

In order to be suitable as a screening assay, a method should be easy to perform, at low cost and without too much risk for the patient. It is also important that the method should have a low false positive rate (high specificity) so that patients who would deliver at term are not unnecessarily treated.

WO 2006/084109 discloses a method for identifying an increased risk of preterm delivery wherein an increase of salivary protease levels is determined. WO 96/12967 provides a method for the early detection of premature delivery comprising measuring the maternal level of corticotrophin-releasing hormone (CRH) in plasma or serum. When plasma CRH is high preterm birth is likely but a low level does not exclude preterm birth.

Despite of all these efforts, there is still a need for reliable biomarkers for the early detection or prediction that a given pregnancy may result in premature delivery.

Pre-eclampsia is a hypertensive disorder that complicates up to 6-8% of pregnancies and remains the leading cause of maternal and perinatal morbidity and mortality (Roberts J M, Cooper D W. Pathogenesis and genetics of pre-eclampsia. Lancet. 2001; 357(9249):53-56. and MacKay A P, Berg C J, Atrash H K. Pregnancy-related mortality from pre-eclampsia and eclampsia. Obstet Gynecol. 2001;97:533-38). Yet, despite extensive research efforts, the etiology of this multi-systematic disorder remains incompletely understood. Vascular endothelial activation followed by vasospasm appears to be the central feature in the pathogenesis of pre-eclampsia.

Theories of its cause include abnormal implantation and development of the placenta, oxidative stress, impaired endothelial prostanoid and nitric oxide homeostasis, genetic polymorphisms, abnormal circulating autoantibodies and an abnormal maternal systematic inflammatory response (Buhimschi I A, Saade G R, Chwalisz K, Garfield R E. The nitric oxide pathway in pre-eclampsia: pathophysiological implications. Hum Reprod Update, 1998;4:25-42, Ward K, Hata A, Jeunemaitre X, Helin C, Nelson L, Namikawa C, Farrington P F, et al. A molecular variant of angiotensinogen associated with pre-eclampsia. Nat Genet 1993;4:59, Wallukat G, Homuth V, Fischer T, Horstkamp B, Jupner A, Baur E, Nissen E, Vetter K, Dudenhausen J W, Haller H, Luft F C. Patients with pre-eclampsia develop agonistic antibodies against the angiotensin AT 1 receptor. J Clin Invest 1999; 103:945-952., Fass M M, Schinling G A, Bailer J F W, Visscher C A, Bakker W W. A new animal model for human pre-eclampsia: ultra-low dose of endotoxin infusion in pregnant rats. Am J Obstet Gynecol 1994; 171: 158-64., Roberts J M, Redman C W. Pre-eclampsia: more than pregnancy induced hypertension. Lancet 1993; 341: 1447-51).

More recently, there has been increased focus on the effects of variations in the expression levels of modulators of angiogenesis, which cause symptoms of pre-eclampsia, including hypertension, proteinuria, endothelial cell activation and increased platelet aggregation (Maynard S E, Min J Y, Merchan J, Lim K H, Li J, Mondal S et al. Excess placental soluble fms-like tyrosine kinase 1 (sFlt1) may contribute to endothelial dysfunction, hypertension, and proteinuria in pre-eclampsia. J Clin Invest 2003; 111: 649-58., Volhard F: Die doppelseitigen haematogenen Nierenerkrankungen Beril, Springer. 1918., Buhimschi I A, Saade G R, Chwalisz K, Garfield R E. The nitric oxide pathway in pre-eclampsia: pathophysiological implications. Hum Reprod Update. 1998; 4:25-42. Takacs P, Kauma S W, Sholley M M, Walsh S W, Dinsmoor M J, Green K. Increased circulating lipid peroxides in severe pre-eclampsia activate NF-kappaB and upregulate ICAM-I in vascular endothelial cells. FASEB J. 2001 ;15:279-81., Lockwood C J, Peters J H. Increased plasma levels of ED1+ cellular fibronectin precede the clinical signs of pre-eclampsia. Am J Obstet Gynecol. 1990;162:358-62.)

Specifically, recent studies have reported that maternal serum concentrations of vascular endothelial growth factor (VEGF), placental growth factor (PIGF) and soluble fins-like tyrosine kinase-1 (sFlt-1) are altered in patients with clinical pre-eclampsia (Levine R J, Maynard S E, Qian C, Lim K H, England U, Yu K F, et al. Circulating angiogenic factors and the risk of pre-eclampsia. N Engl J Med 2004:12;350:672-83., Maynard S E, Min J Y, Merchan J, Lim K H, Li J, Mondal S et al. Excess placental soluble fins-like tyrosine kinase 1 (sFlt1) may contribute to endothelial dysfunction, hypertension, and proteinuria in pre-eclampsia. J Clin Invest 2003; 111 :649-58., Levine R J, Maynard S E, Qian C, Lim K H, England U, Yu K F, et al. Circulating angiogenic factors and the risk of pre-eclampsia. N Engl J Med 2004: 12; 350: 672-83.)

Neurologic manifestations are common in severe pre-eclampsia, and include seizures or coma (eclampsia), stroke, hypertensive encephalopathy, headaches, and visual aberrations. Despite intensive research efforts, the etiology of pre-eclampsia and its neurologic manifestations remains unknown.

Pre-eclampsia can vary in severity from mild to life threatening. A mild form of pre-eclampsia can be treated with bed rest and frequent monitoring. For moderate to severe cases, hospitalization is recommended and blood pressure medication or anticonvulsant medications to prevent seizures are prescribed.

The symptoms of pre-eclampsia typically appear after the 20th week of pregnancy and are usually detected by routine monitoring of the woman's blood pressure and urine. However, these monitoring methods are ineffective for diagnosis of the syndrome at an early stage, which could reduce the risk to the subject or developing fetus, if an effective treatment were available.

Currently, there are no known cures for pre-eclampsia. The only definitive treatment for pre-eclampsia is delivery of the fetus and placenta. Hospitalization, strict bed rest, magnesium sulfate administration to prevent convulsions, and prompt delivery remain as the current standard of therapy for pre-eclampsia.

Currently also, there is no single test to predict or diagnose pre-eclampsia or to predict the severity of the condition that will develop in a particular patient. Early symptoms include persistent headaches, blurred vision or sensitivity to light and abdominal pain. However, a diagnosis of pre-eclampsia is not typically made until increased blood pressure and protein in the urine (proteinuria) are revealed, typically in routine physician tests following the 20th week of pregnancy. Severe effects of pre-eclampsia, including seizures, cerebral hemorrhage, disseminated intravascular coagulation and renal failure, may appear very shortly following such diagnosis. These methods are imprecise and provide little insight into the likelihood of the most severe symptoms developing. Moreover, the current diagnostics require physician oversight and invasive methodologies, further delaying and complicating early and immediate assessment.

Recently, a number of diagnostic methods for determing pre-eclampsia have been described. WO 90/07122 describes the diagnosis of pre-eclampsia using an assay to measure a mitogenic factor in blood. This assay incorporates the culturing of animal fibroblasts or smooth muscle cells and is therefore a time consuming and elaborate method.

U.S. Pat. No. 5,849,474 discloses a method for the diagnosis of pre-eclampsia wherein the levels of a hemoglobin variant and a red blood cell glycolytic enzyme are measured. It remains unclear whether this method may be used to identify women at risk for pre-eclampsia.

WO 02/04678 provides methods for diagnosing pre-eclampsia wherein the levels of syncytin mRNA expression or polypeptide are measured.

WO 2005/093413 provides a method for the diagnosis of pre-eclampsia wherein the level of free hemoglobin is measured in a cerebrospinal fluid sample.

US patent application No. 2005/0255114 describes a proteomics approach towards the identification of patients at risk of pre-eclampsia. It provides a number of 852 candidate proteins that may exhibit either elevated or decreased levels of expression in patients with pre-eclampsia.

WO 2005/078128 is directed towards a method for determining whether a subject is at increased risk for pre-eclampsia by detecting the presence or absence of a variant genotype of the DDAH gene in a biological sample.

US patent application No. 2005/0074746 relates to a method for determining if a pregnant womam is at risk of developing pre-eclampsia wherein human trophoblast cells are cultured in the presence of serum or plasma of a pregnant woman.

WO 2006/069373 teaches a method of diagnosing a pregnant woman as having or being susceptible to developing a hypertensive disorder. This method involves the measurement of levels of soluble fms-like tyrosine kinase 1 in a urine sample.

Despite of all the above efforts, there is an urgent need for an early and accurate method for the detection and diagnosis of pre-eclampsia and associated proteinuric hypertensive disorders.

Low birth weight includes two pathologic conditions and one normal condition. The normal condition refers to the healthy but constitutionally small baby which is often referred to as Small for Gestational Age (SGA). The pathologic conditions include preterm delivery and intrauterine growth retardation (IUGR). Synonymous terms found in the literature to describe infants with IUGR include intrauterine growth restriction and foetal growth retardation. In the United States, IUGR is linked to an increase of six to 10 times in perinatal mortality (Bernstein I, Gabbe S G. Intrauterine growth restriction. In: Labbe S G, Niebyl J R, Simpson J L, Annas G J, eds. Obstetrics: normal and problem pregnancies. 3rd ed. New York: Churchhill-Livingstone, 1996;863-86, Creasy R K, Resnik R. Intrauterine growth restriction. In: Creasy R K, Resnik R, eds. Maternal-foetal medicine: principles and practice. 3d ed. Philadelphia: Saunders, 1994;558-74).

The current WHO criterion for low birth weight is a weight less than 2,500 g (5 lb, 8 oz) or below the 10th percentile for gestational age (Dunn P M. The search for perinatal definitions and standards. Acta Paediatr Scand Suppl 1985;319:7-16.).

Intrauterine growth retardation (IUGR) is defined as the condition in which foetal growth is delayed or arrested and the actual foetal growth is less than the natural and latent growth corresponding to the gestation age. The condition is caused by foetal, placental and maternal factors, and these three factors coexist in most cases. Concrete causes of IUGR include foetal chromosomal aberration, intrauterine infections and abnormal metabolism, which induce the symmetrical type of IUGR in which the same degree of growth retardation is observed in the head, stature and trunk. Other concrete causes of IUGR include maternal complications such as gestational toxicosis and chronic renal diseases, and disorders of the uteroplacental circulation due to placental or umbilical abnormalities, which induce the asymmetrical type of IUGR in which the growth of head and stature is within normal ranges, while that of trunk is delayed. IUGR may result in significant foetal morbidity and mortality if not properly diagnosed. When IUGR is recognized, it is important to attempt to correct reversible causes, although many of the conditions responsible for IUGR are not amenable to antenatal therapy. Close foetal surveillance with delivery before 38 weeks of gestation is usually recommended. Some infants born with IUGR have cognitive and medical problems, although for most infants the long-term prognosis is good. However it is now widely accepted that infants born small have an increased likelihood of many adult diseases including early onset hypertension, obesity, diabetes and ischaemic heart disease (Barker, D. J. (1995). “Intrauterine programming of adult disease.” Mol Med Today 1(9): 418-23).

Small for gestational age (SGA) describes an infant's size at birth compared to appropriate population standards and is not synonymous with IUGR which requires antenatal foetal auxology that demonstrates subnormal prenatal growth velocity. Infants born following IUGR may or may not be SGA. The definition of SGA is arbitrary, but in growth clinics SGA is commonly defined as birth weight and/or length two or more standard deviation scores below the mean for gender and gestation, which is consistent with the definition of childhood short stature. As birth length is not always available and is often not as reliably measured as weight, SGA is most commonly defined by birth weight alone.

The known methods for diagnosing the progress of pregnancy, have the disadvantage of detecting the status of pregnancy disorders at a relatively late stage, when the clinical signs and symptoms are already apparent.

Before the development of ultrasonography, delayed foetal growth was indicated by low maternal weight gain, Leopold maneuvers and fundal height measurement. Currently, IUGR is still often suspected on the basis of fundal height measurements. A significant lag in fundal height is a 4 cm or greater difference than expected for gestational age. However, even carefully performed fundal height measurements only have a 26 to 76 percent sensitivity in predicting IUGR (Calvert J P, Crean E E, Newcombe R G, Pearson J F. Antenatal screening measurements by symphysis-fundal height. BMJ 1982; 285: 846-9. 12).

IUGR is frequently detected in a pregnancy with a less-than-expected third-trimester weight gain (100 to 200 g per week) or as an incidental finding on ultrasound examination when foetal measurements are smaller than expected for gestational age.

The main prerequisite for determining IUGR is precise dating. The most accurate dating method uses ultrasound examination at eight to 13 weeks. Later ultrasound examinations are helpful, but the margin of error is increased. The date of the last menstrual period, early uterine sizing and detection of foetal heart tones are helpful ways to accurately date the pregnancy. Most cases of IUGR present during the third trimester, which makes them difficult to accurately diagnose. This is especially true if the patient has presented for prenatal care at a late stage. The physician must determine if the dating is incorrect and the foetal size is actually normal or if the mother truly needs further evaluation for IUGR.

When the suspicion of IUGR is strong, a complete assessment of maternal risk factors is indicated. This includes past medical and obstetric history, medication use, recent infections, occupational or toxic exposures, and a history of tobacco, alcohol or illicit drug use.

Ultrasonography is normally the first study done to assess IUGR. This test loses its accuracy as the pregnancy progresses, but the sensitivity and positive predictive value can be improved if several variables are combined (Doubilet P M, Benson C B. Sonographic evaluation of intrauterine growth retardation. AJR Am J Roentgenol 1995; 164: 709-17). These variables include estimated foetal weight, head circumference and abdominal circumference.

Estimated foetal weight is the most common screen. It is based on the measurements of head circumference, abdominal circumference and femur length. These measurements are plotted on a preexisting standardized chart. In about 95 percent of cases, ultrasound examination allows an estimation of foetal weight with a 15 to 18 percent variance (Doubilet P M, Benson C B. Sonographic evaluation of intrauterine growth retardation, Am. J. Roentgenol. 1995; 164: 709-717). An estimated foetal weight of less than the sixth percentile strongly correlates with growth retardation, and an estimated foetal weight of greater than the 20th percentile virtually rules out IUGR. An estimated foetal weight at the 15th percentile or less, or a decreasing estimated foetal weight as determined by serial ultrasound examination, is suggestive of IUGR.

In all growth-retarded foetuses, the abdominal circumference is the first biometric measure to change. This translates to an increased ratio of head circumference to abdominal circumference. The ratio of head circumference to abdominal circumference is normally one at 32 to 34 weeks and falls below one after 34 weeks. A ratio of greater than one detects about 85 percent of growth-restricted fetuses (Hadlock F. Ultrasound evaluation of foetal growth. In: Callen P, ed. Ultrasonography in obstetrics and gynecology. 3rd ed. Philadelphia: Saunders, 1994; 129-42).

There are several hormone assays suggested to give an indication whether placental function is normal or to predict impending foetal death. Among those tests are: urine estriol, urine total estrogens, serum unconjugated estriol and serum placental lactogen.

Estriol is an estrogenic compound produced by the placenta from precursors derived from foetal adrenal cortex and foetal liver. The conjugated form of estriol is excreted into the maternal urine. Serum estriol can be measured either as total estriol or as unconjugated estriol. It usually is measured as unconjugated estriol-in order to exclude a maternal contribution to the conjugated fraction. Urine estriol can be measured as total estriol or as total estrogens, since estriol normally constitutes about 90% of urine total estrogens. Estriol can be detected by immunoassay as early as the ninth week of gestation. Thereafter, estriol values slowly but steadily increase until the last trimester, when there is a more pronounced increase. Clinical use of estriol measurement is based on the fact that severe acute abnormality of the fetoplacental unit, such as a dead or dying placenta, is manifested either by failure of the estriol level to continue rising or by a sudden marked and sustained decrease in the estriol level. M. Scharf etal. (J. Obstet. Gynec. reprod. Biol., 17:365-75, 1984) conclude that in view of the low correlation between patients with abnormal serum free estriol as the antepartum pathological test, the estriol measurement can not be considered a reliable predicting tool to estimate the actual pregnancy outcome.

Urine total estrogen was the first test used, since total estrogen can be assayed by standard clinical techniques. However, urine glucose falsely increases the results and certain other substances such as urobilinogen also may interfere. On the other hand, maternal hypertension, preeclampsia, severe anemia and impaired renal function can decrease considerably urine estrogen or estriol secretion. Decrease in the level may also occur to a variable degree in a number of fetuses with severe congenital anomalies. It was also reported that continued bed-rest to the pregnant woman caused an increase in the estriol excretion values of about 20 to 30% over the levels determined from ambulatory persons. Because of the problems associated with collection of urine or serum estriol specimens and interpretation of the values, as well as the disturbing number of false positive and negative test results, most of the clinical people refrain from correlating these measurements with placental disorders. Moreover, all the previous methods did not reveal the pregnancy disorders at an early stage of their initiation, but only when the particular disease was already apparent.

More recently, another method for the detection of pregnancy disorders including IUGR was disclosed. U.S. Pat. No. 5,198,366 to Silberman describes that abnormal levels of a human placental protein designated PP-13 are indicative for IUGR.

WO99/43851 describes a method for diagnosing susceptibility to IUGR by determining abnormalities in the HLA-G gene, gene expression or protein.

### SUMMARY OF THE INVENTION

Broadly stated, the present invention relates to the finding that levels of particular biomarkers can be measured to provide an indication of the risk of preterm delivery and/or certain pregnancy associated conditions. Determination of the risk can allow early medical intervention for the benefit of the patient and/or the foetus or newborn. Alternatively, a patient identified to be at risk can be monitored so that appropriate steps or treatment can be taken should it become necessary as the pregnancy progresses.

In an aspect of the invention there is provided a method for identifying a woman with an increased risk of premature delivery, comprising the steps of:

(a) obtaining a biological sample from a pregnant patient after week 12 and prior to week 37 of pregnancy;

(b) determining the level of at least one biomarker in the sample, the biomarker being selected from the group consisting of transferrin, inter-alpha trypsin inhibitor heavy chain H4, apolipoprotein B48 receptor, alpha-1B-glycoprotein, antithrombin III and apolipoprotein A-IV precursor;

(c) comparing the level of the at least one biomarker with a level characteristic of normal pregnancy; and

(d) determining whether the patient has an increased risk of premature delivery from the comparison.

In another aspect there is provided a method for preventing a preterm delivery comprising the steps of identifying a woman with an increased risk of premature delivery as described above, and treating the woman with a tocolytic agent.

In another aspect of the invention there is provided a method for improving the life expectancy of a newborn by ameliorating the effects of a preterm delivery, comprising the steps of identifying a woman with an increased risk of premature delivery as described above, and treating the woman with a steroid or another effective medication in order to promote foetal lung maturation or to delay delivery with treatment such as administration of progesterone (da Fonseca E B, Bitter R E, Carvalho M H, Zugaib M. Prophylactic administration of progesterone by vaginal suppository to reduce the incidence of spontaneous preterm birth in women at increased risk: a randomized placebo-controlled double-blind study. Am J Obstet Gynecol. 2003; 188(2):419-424 and Meis P J, Klebanoff M, Thom E. et al. Prevention of recurrent preterm delivery by 17 alpha-hydroxyprogesterone caproate. National Institute of Child Health and Human Development Maternal-Fetal Medicine Units Network. NEJM. 2003; 348(24):2379-85.)

In another aspect of the invention there is provided a method for determining whether a given pregnancy is likely to proceed to full term, comprising the steps of:

(a) obtaining a biological sample from a pregnant patient after week 12 and prior to week 37 of pregnancy;

(b) determining the level of at least one biomarker in the sample, the biomarker being selected from the group consisting of transferrin, inter-alpha trypsin inhibitor heavy chain H4, apolipoprotein B48 receptor, transferrin, alpha-1B-glycoprotein, antithrombin III and apolipoprotein A-IV precursor;

(c) comparing the level of the at least one biomarker with a level characteristic of normal pregnancy; and

(d) determining whether the pregnancy of the patient is likely to proceed to term from the comparison.

The invention in at least some forms also relates to methods for determining or aiding in the determination that a pregnant woman is at risk of developing pre-eclampsia or other hypertensive disorder(s). In certain embodiments, the invention provides methods for determining or aiding in the determination that a pregnant woman has pre-eclampsia. In other embodiments, the invention provides for methods of screening or pre-screening pregnant women to identify those pregnant women with a low risk of developing hypertensive disorders, which reduces the need for additional testing throughout the pregnancy.

Accordingly, in another aspect of the invention there is provided a method for identifying a woman with an increased risk of pre-eclampsia or developing a hytertensive disorder associated with pregnancy, comprising the steps of:

(a) obtaining a biological sample from a pregnant patient after week 12 and prior to week 37 of pregnancy;

(b) determining the level of at least one biomarker in the sample, the biomarker being selected from the group consisting of transferrin, alpha-1B-glycoprotein, antithrombin III, haptoglobin and transthyretin;

(c) comparing the level of the at least one biomarker with a level characteristic of normal pregnancy: and

(d) determining whether the patient has an increased risk of pre-eclampsia or developing a hypertensive disorder from the comparison.

In another aspect of the invention there is provided a method for ameliorating the effects of a preterm delivery for the newborn, comprising the steps of identifying a woman with an increased risk of pre-eclampsia as described above, and treating the woman with a steroid or another effective medication in order to promote foetal lung maturation.

In still another aspect of the invention there is provided a method for screening or pre-screening pregnant women to identify those pregnant women with a low risk of developing a hypertensive disorder, comprising the steps of:

(a) obtaining a biological sample from a pregnant patient after week 12 and prior to week 37 of pregnancy;

(b) determining the level of at least one biomarker in the sample, the biomarker being selected from the group consisting of transferrin, alpha-1B-glycoprotein, antithrombin III, haptoglobin and transthyretin;

(c) comparing the level of the at least one biomarker with a level characteristic of normal pregnancy; and

(d) determining whether the patient is at low risk of developing a hypertensive disorder from the comparison.

The invention in at least some forms also relates to methods for determining or aiding in the determination that a pregnant woman is at risk of developing IUGR or other foetal growth related disorder(s). In certain embodiments, the invention provides methods for determining or aiding in the determination that a pregnant woman has IUGR. In other embodiments, the invention provides methods of screening or pre-screening pregnant women to identify those pregnant women with a low risk of developing IUGR, which reduces the need for additional testing throughout the pregnancy.

In another aspect of the invention there is provided a method for identifying a woman with an increased risk of IUGR, comprising the steps of:

(a) obtaining a biological sample from a pregnant patient after week 12 and prior to week 37 of pregnancy;

(b) determining the level of at least one biomarker in the sample, the biomarker being selected from the group consisting of transferrin, inter-alpha trypsin inhibitor heavy chain H4, antithrombin HI, haptoglobin and transthyretin;

(c) comparing the level of the at least one biomarker with a level characteristic of normal pregnancy; and

(d) determining whether the patient has an increased risk of IUGR from the comparison.

In another aspect of the invention there is provided a method for ameliorating the effects of a preterm delivery for the newborn, comprising the steps of identifying a woman with an increased risk of IUGR as described above, and treating the woman with a steroid or another effective medication in order to promote foetal lung maturation.

In yet another aspect of the invention there is provided a method for screening or pre-screening pregnant women to identify those pregnant women with a low risk of developing IUGR, comprising the steps of:

(a) obtaining a biological sample from a pregnant patient after week 12 and prior to week 37 of pregnancy;

(b) determining the level of at least one biomarker in the sample, the biomarker being selected from the group consisting of transferrin, inter-alpha trypsin inhibitor heavy chain H4, antithrombin III, haptoglobin and transthyretin;

(c) comparing the level of the at least one biomarker with a level characteristic of a normal pregnancy; and

(d) determining whether the patient has a low risk of developing IGUR from the comparison.

The features and advantages of the invention will become further apparent from the following detailed description of non-limiting embodiments.

### BRIEF DESCRIPTION OF THE ACCOMPANYING DRAWINGS

FIG. 1: Diagrams illustrating the statistical Analysis of 2D-DICE Gels. (A) Following imaging, protein spots labelled with either Cy3 or Cy5 are standardised against the pooled internal control spot labelled with Cy2. Gel to gel variation is removed as the samples have all been resolved in the same gel. The analysis is performed utilizing the DIA module of DeCyder™ 2-D Differential Analysis Software. (B) The internal standard is also used for matching and comparing protein spot abundance across gels (performed by the DeCyder™ BVA module). This enables the statistical comparison of protein abundance changes in multiple samples.

FIG. 2: Graph showing transferrin levels for 16 singleton pregnancies, obtained in an ELISA assay.

FIG. 3: Graph showing transferrin levels for 4 normal pregnancies obtained utilizing an ELISA assay.

FIG. 4: Graph showing transferrin levels estimated at 26 weeks of pregnancy derived from the regression analysis of FIG. 2.

FIG. 5: Graph showing transferrin estimated rate of change at 26 weeks of pregnancy.

FIG. 6: Graph showing haptoglobin levels for 15 singleton pregnancies obtained utilizing an ELISA assay.

FIG. 7: Graph showing haptoglobin levels for 4 normal pregnancies obtained utilizing an ELISA assay.

### DETAILED DESCRIPTION OF EMBODIMENTS OF THE INVENTION

The invention relates to a screening assay which can provide an early, biochemical indication of increased risk of preterm delivery. The method may, for instance, provide an indication of impending preterm delivery as early as three to four months prior to delivery, 2 or 1 month, or 4, 3. 2 or 1 week prior to delivery. As such the method may allow early intervention in the course of preterm delivery, and provides an additional factor which can indicate those pregnancies at greatest risk.

The method comprises obtaining a biological sample from the pregnant patient after about week 12 of pregnancy and prior to about week 36 or 37, and determining the level of one or more biomarkers as described herein in the sample. The presence of an elevated level of the biomarker in the sample indicates a patient who is at risk for preterm delivery.

In particular, the invention provides a method for identifying a woman with an increased risk of premature delivery, comprising the steps of obtaining a biological sample from a pregnant patient after week 12 and prior to week 37 of pregnancy and in the biological sample, determining the level of at least one biomarker selected from the group consisting of transferrin, inter-alpha trypsin inhibitor heavy chain H4, apolipoprotein B48 receptor, alpha-1B-glycoprotein, antithrombin III and apolipoprotein A-IV precursor, and comparing the level of the at least one biomarker with a level characteristic of normal pregnancy that proceeds to term, wherein an abnormal level of the at least one biomarker is indicative of an increased risk of preterm delivery.

Hence, the method can determine impending delivery from very early in gestation through week 36 or 37. Deliveries prior to 20 weeks gestation are generally called spontaneous abortions rather than preterm deliveries. The present method can be used to detect spontaneous abortions (12 to 20 weeks gestation) and preterm deliveries (20 to 37 weeks gestation). Term pregnancies are from 37 to 40 weeks.

In particular, in contrast to most prior art methods, the method is capable of predicting a preterm delivery months in advance, whereas the prior art provides assays that may be used when delivery is imminent, i.e. within a week or two.

The term “abnormal level” in the context of diagnosing preterm delivery is meant to indicate either a decreased level of transferrin, inter-alpha trypsin inhibitor heavy chain H4, apolipoprotein B48 receptor or apolipoprotein A-IV precursor or an increased level of alpha-1B-glycoprotein or antithrombin III.

Premature delivery or preterm delivery or preterm birth are used interchangeably herein and are herein defined as birth after week 20 but before 37 completed weeks of pregnancy and may occur due to several different aetiologies.

A significant problem for premature babies is lung immaturity. Foetal lungs can be matured by the administration of beta methasone. However, if preterm delivery is likely, the beta methasone needs to be administered quickly in order to permit lung maturation to occur prior to delivery.

A method of determining that delivery is imminent and that consequently tocolytic therapy may not succeed can improve patient care by facilitating clinical decision making with regard to administration of agents such as steroids to mature foetal lungs. This treatment can be undertaken as a priority followed by a decision regarding persevering with tocolytic therapy. Hence, in one or more embodiments, the invention further relates to a method for ameliorating the effects of a preterm delivery for the newborn, comprising the steps of identifying a woman with an increased risk of premature delivery as described herein, and treating the woman with a steroid or another effective medication in order to promote foetal lung maturation. Alternatively, progersterone may be administered to women determined to be at high risk in an attempt to prevent preterm birth from occurring.

Embodiments of methods as described herein may also be utilized to provide an indication that any given pregnancy will proceed to term, if the level of the biomarker measured is within the normal range.

The invention also relates to a screening assay which provides an early, biochemical indication of increased risk of pre-eclampsia. The method may, for example, provide an indication of impending pre-eclampsia as early as three to four months prior to delivery, such as 2 or 1 month or 4, 3, 2 or 1 week prior to delivery. As such the method may allow early intervention in the course of pre-eclampsia, and provides an additional factor which can indicate those pregnancies at greatest risk.

This method comprises obtaining a biological sample from the pregnant patient after about week 12 of pregnancy and prior to about week 36 or 37 and determining the level of one or more biomarkers as disclosed herein in the sample. The presence of an abnormal level of the biomarker in the sample indicates a patient who is at risk for pre-eclampsia.

In particular, in another form, the invention provides a method for identifying a woman with an increased risk of pre-eclampsia comprising the steps of obtaining a biological sample from a pregnant patient after week 12 and prior to week 37 of pregnancy and in the biological sample, determining the level of at least one biomarker selected from the group consisting of transferrin, alpha-1B-glycoprotein, antithrombin III, haptoglobin and transthyretin and comparing the level of the at least one biomarker with a level characteristic of pregnancies that proceed to term, wherein an abnormal level of the at least one biomarker is indicative of an increased risk of pre-eclampsia.

In contrast to prior art methods, one or more embodiments of this method may allow the prediction of pre-eclampsia months in advance, whereas the prior art provides assays that may be used when clinical symptoms of pre-eclampsia are already apparent.

As used herein, the term “pre-eclampsia” is defined according to criteria established by the committee on Terminology of the American College of Obstetrics and Gynecology, such as a blood pressure of at least 140/90 mm Hg and urinary excretion of at least 0.3 grams of protein in a 24-hour urinary protein excretion (or at least +1 or greater on dipstick testing), each on two occasions 4-6 hours apart. The term pre-eclampsia is used so as to include severe pre-eclampsia.

As used herein, “severe pre-eclampsia” is also defined in accordance with established criteria, as a blood pressure of at least 160/110 mm Hg on at least 2 occasions 6 hours apart and greater than 5 grams of protein in a 24-hour urinary protein excretion or persistent+3 proteinuria on dipstick testing. Severe pre-eclampsia may include HELLP syndrome (hemolysis, elevated liver enzymes, low platelet count). Other elements of severe preeclampsia may include in-utero growth restriction (IUGR) in less than the 10% percentile according to the US demographics, persistent neurologic symptoms (headache, visual disturbances), epigastric pain, oliguria (less than 500 mL/24 h), serum creatinine greater than 1.0 mg/dL, elevated liver enzymes (greater than two times normal), thrombocytopenia (<100,000 cells/[mu]L).

The term “abnormal level” in the context of diagnosis of pre-eclampsia is meant to indicate either a decreased level of transthyretin or an increased level of transferrin, haptoglobin, alpha-1B-glycoprotein or antithrombin III.

When (severe) pre-eclampsia is diagnosed according to a method as described above, immediate delivery may be indicated. The chances for survival of the foetus increase when at that point in time effective medication is supplied to promote foetal lung maturation. The invention is, therefore, also directed to a method for ameliorating the effects of a preterm delivery for the newborn, comprising the steps of identifying a woman with an increased risk of pre-eclampsia as decribed herein and treating the woman with a steroid or another effective medication in order to promote foetal lung maturation.

Since the methods according to the invention also have a good negative predictive value, the invention is also directed towards a method for screening or pre-screening pregnant women to identify those pregnant women with a low risk of developing hypertensive disorders, the method comprising the steps of obtaining a biological sample from a pregnant patient after week 12 and prior to week 37 of pregnancy and in the biological sample. determining the level of at least one biomarker selected from the group consisting of alpha-1B-glycoprotein, antithrombin III and transthyretin, and comparing the level of the at least one biomarker with a level characteristic of pregnancies that proceed to term, wherein a normal level of said at least one biomarker is indicative for a pregnancy with a low risk of developing hypertensive disorders.

Birth weight is herein defined as the weight of a baby at its birth. It has direct links with the gestational age at which the child was born and can be estimated during the pregnancy by measuring fundal height. A baby born within the normal range of weight for that gestational age is known as appropriate for gestational age (AGA). Those born above or below that range have often had an unusual rate of development—this often indicates complications with the pregnancy that may affect the baby or its mother.

The incidence of birth weight being outside of the AGA is influenced by the parents in numerous ways, including genetics, the health of the mother, particularly during the pregnancy, and environmental factors. There have also been numerous studies that have attempted, with varying degrees of success, to show links between birth weight and later-life conditions, including diabetes, obesity, tobacco smoking and intelligence.

Large for gestational age (LGA) is herein defined as babies whose birth weight lies above the 90th percentile for that gestational age. Macrosomia, also known as big baby syndrome, is sometimes used synonymously with LGA, or is otherwise defined as a fetus that weighs above 4000 to 4500 grams regardless of gestational age.

Small for gestational age (SGA) is herein defined as babies whose birth weight lies below the 10th percentile for that gestational age. They may or may not have been the subject of intrauterine growth retardation (IUGR). Low birth weight, is sometimes used synonymously with SGA, or is otherwise defined as a fetus that weighs less than 2500 g regardless of gestational age.

There is a 4-8% incidence of low birth weight in developed countries, and 6-30% in developing countries. Much of this can be attributed to the health of the mother during pregnancy. One third of babies born with a low birth weight are also small for gestational age. SGA is generally diagnosed by measuring the mother's uterus, with the fundal height being less than it should be for that stage of the pregnancy. If it is suspected, the mother will usually be sent for an ultrasound to confirm.

There are two distinct categories of growth retardation, indicating the stage at which the development was slowed. Small for gestational age babies can be classified as having symmetrical or assymmetrical growth retardation.

Symmetrical growth retardation, less commonly known as global growth retardation, indicates that the fetus has developed slowly throughout the duration of the pregnancy and was thus affected from a very early stage. The head circumference of such a newborn is in proportion to the rest of the body. Common causes include early intrauterine infections, such as cytomegalovirus, rubella or toxoplasmosis, chromosomal abnormalities, maternal substance abuse, such as fetal alcohol spectrum disorder.

Asymmetrical growth retardation occurs when the embryo/fetus has grown normally for the first two trimesters but encounters difficulties in the third, usually pre-eclampsia. Such babies have a disparity in their length and head circumference when compared to the birth weight. A lack of subcutaneous fat leads to a thin and small body that is out of proportion with the head. Other symptoms include dry, peeling skin and an overly-thin umbilical cord, and the baby is at increased risk of hypoxia and hypoglycaemia. Possible treatments include the early induction of labour, though this is only done if the condition has been diagnosed and seen as a risk to the health of the fetus.

The invention, in another form, also relates to a screening assay which can provide an early, biochemical indication of increased risk of IUGR or an IUGR related disorder. The method, may for instance, provide an indication of impending IUGR as early as three to four months prior to delivery, or 2 or 1 month, or 4, 3, 2 or 1 week prior to delivery. As such the method may allow early intervention in the course of MGR and provide an additional factor which can indicate those pregnancies at greatest risk.

In particular, the invention provides a method for identifying a woman with an increased risk of IUGR or an IUGR related disorder, comprising the steps of obtaining a biological sample from a pregnant patient after week 12 and prior to week 37 of pregnancy and in the biological sample, determining the level of at least one biomarker selected from the group consisting of transferrin, inter-alpha trypsin inhibitor heavy chain H4, antithrombin III, haptoglobin and transthyretin, and comparing the level of the at least one biomarker with a level characteristic of pregnancies that proceed to term, wherein an abnormal level of the at least one biomarker is indicative for an increased risk of IUGR.

In contrast to the prior art methods, one or more embodiments of this method may allow the early prediction of IUGR months in advance, whereas the prior art provides assays that may be used when clinical symptoms of IUGR (eg., advanced stage placental illness) are already apparent.

IUGR is herein defined as a condition wherein the newborn has a birthweight of less than the 10^{th} percentile combined with a pathological condition that inhibits expression of the normal intrinsic growth potential.

The term “abnormal level” in the context of diagnosing IUGR is meant to indicate either a decreased level of transferrin, inter-alpha trypsin inhibitor heavy chain haptoglobin or transthyretin or an increased level of antithrombin III.

When IUGR is diagnosed according to a method as described above, premature delivery may be indicated. As also described above, the chances for survival of the foetus increase when at that point in time effective medication is supplied to promote foetal lung maturation. The invention, therefore, is also directed to a method for ameliorating the effects of a preterm delivery for the newborn, comprising the steps of identifying a woman with an increased risk of IUGR as described herein and treating the woman with a steroid or another effective medication in order to promote foetal lung maturation.

Since the methods embodied by the invention are useful in predicting increased risk of IGUR, they also have a good negative predictive value. Hence, the invention also relates to a method for screening or pre-screening pregnant women to identify those pregnant women with a low risk of developing IUGR or IUGR related disorders, the method comprising the steps of obtaining a biological sample from a pregnant patient after week 12 and prior to week 37 of pregnancy and in the biological sample, determining the level of at least one biomarker selected from the group consisting of transferrin, inter-alpha trypsin inhibitor heavy chain H4, antithrombin haptoglobin and transthyretin, comparing the level of the at least one biomarker with a level characteristic of pregnancies that proceed to term, wherein a normal level of said at least one biomarker is indicative for a pregnancy with a low risk of developing hypertensive disorders.

The biological sample used in a method embodied by the invention may be any suitable sample obtained from a pregnant woman. The results presented herein are based on sera obtained from pregnant women. Other biological fluids that may be used include amniotic fluid, vaginal excretions, saliva, and urine. Typically, however, the biological sample is a blood, plasma or serum sample. It is a great advantage when a screening assay can be performed on a blood, serum or plasma sample since these samples can be taken from a patient with minimal discomfort.

The levels of the particular biomarker assayed are typically compared with the levels of that same biomarker in the same type of sample taken from a number of patients that experienced term delivery. Most typically, the control samples are from a normal pregnancy of the same gestational period or age as the period or age of the patient being screened. For higher precision, the samples can be age matched.

The term “normal pregnancy” is meant to indicate a pregnancy that proceeds to term without any complications. Usually, a normal pregnancy is a pregnancy without, such as in particular, preterm delivery, pre-eclampsia or a growth retardation of the foetus. A normal pregnancy as defined herein ends with the delivery of an AGA newborn.

It will be understood that the level of the biomarker(s) as described herein can be measured directly or indirectly by a variety of different techniques. However, if the method is to be used as a screening assay, an immunological method is preferred. Antibodies against the biomarkers are readily available. A person skilled in the art will also know how to design and develop an immunological assay based on such antibodies. For at least some of the markers, commercial assays are available. Particularly preferred immunological methods are radioimmunoassays and enzyme-linked immunosorbent assays (ELISAs) because they are particularly amenable to routine laboratory practice.

Rather than the intact biomarker(s), natural metabolites, degradation products or artificial derivatives thereof such as may be obtained by enzymatic cleavage, chemical modification (eg., addition of side chain(s) or the coupling of reporter groups for detection purposes) or other treatments, and which correlate with the level of the intact form of the marker, may be measured.

### Patients to be Tested

Methods embodied by the invention may be used on any pregnant woman following about 12 weeks gestation and prior to term pregnancies (week 36 or 37). In addition to screening any pregnant woman to determine whether delivery is imminent, the patients who are preferably screened are those patients with clinically intact membranes in a high risk category for preterm delivery, and more preferably, all those women whose pregnancies are not sufficiently advanced to ensure delivery of a healthy foetus. Ninety percent of the foetal morbidity and 100 percent of the foetal mortality associated with preterm delivery is for those foetuses delivered prior to 32 to 34 weeks gestation.

Therefore, 32 to 34 weeks gestation is an important cut-off for the health of the foetus, and preferably women whose pregnancies are at least about 12 weeks and prior to 34 weeks in gestation are tested.

In addition, there are a large number of factors known to be associated with the risk of preterm delivery. Those factors include multiple foetus gestations; smoking, incompetent cervix; uterine anomalies; polyhydramnios; nulliparity; previous preterm rupture of membranes or preterm labor; preeclampsia; first trimester vaginal bleeding; little or no antenatal care; and symptoms such as abdominal pain, low backache, passage of cervical mucus and contractions. Any pregnant woman at 12 or more weeks gestation with clinically intact membranes and having one or more of these risk factors for preterm delivery is preferably tested throughout the risk period; i.e., until about week 34 to 37.

Samples taken in the second trimester of pregnancy are desirably utilized in methods embodied by the invention. The samples should be tested immediately after preparation. Storage up to several days under appropriate conditions was shown not to affect the results. Nevertheless, addition of protein stabilisers and protease inhibitors such as kallikrein inhibitors (PMSF) is recommended.

Pregnancy associated hypertensive disorders that may be detected by a method embodied by the invention include hypertensive disorders with our without associated proteinuria, chronic hypertension, pre-eclampsia and gestational hypertension (pregnancy induced hypertension (PIH)). Hypertensive disorders during pregnancy are generally associated with a diastolic blood pressure of 90 mm Hg or more. Any suitable treatment or medicament can be administered for treatment of the pregnancy associated hypertensive disorder, and may for example be selected from aldomet, labatolol, hydralazine, nifedipine (Procardia, Adalat), diuretics, clonidine, calcium channel blockers, vasodilators, magnesium sulphate (MgSO₄), and combinations thereof.

Tocolytic agents that may be utilised in methods as described herein may for example be selected from MgSO₄, nifredipine, fenoterol, ritodrine (Yutopar), atosiban, salbutamol, indomethacin, terbutaline (Brethine). oxcytosin antagonists, and combinations thereof.

Steroids that may be used for promoting maturation of foetal lungs include corticosteroids and glucocorticoids (such as betamethasone, dexamethasone, and hydrocortisone).

### Assay Procedures

Abnormal levels of Inter-alpha trypsin Inhibitor heavy chain H4 (ITI H4) were found to be indicative of the diagnosis of preterm delivery. On average, patients with term delivery exhibited a level of ITI H4 that was about 2 times higher as compared to patients with preterm delivery.

Levels of ITI H4 were measured using two dimensional differential gel electrophoresis (2D-DIGE) as described in the examples. 2D-DIGE is a powerful tool to enable simultaneous visualization of relatively large portions of the proteome (Marouga et al., Anal. Bioanal. Chem. 2005. 382:669-78). For the routine determination of ITI H4 in the clinical laboratories, an immunological method is preferred. Such methods are further detailed in example 10.

The molecular cloning of ITI H4 has been described in Saguchi K. Et al., J. Biochem. 119: 898-905 (1996). This allows the expression of recombinant ITI H4 and the generation of monoclonal and polyclonal antibodies against this marker protein (see Examples 8 and 9). Moreover, methods for the quantitative determination of inter-alpha trypsin inhibitors have been described. In particular, EP 0764849 A1 describes an immunological ELISA for the determination of ITI H1 and H2 whereas WO 01/63280 describes the development of a monoclonal antibody specific for ITI. Moreover, Choi-Miura describes a quantitative ELISA for the measurement of IHRP in human plasma which is believed to be identical with ITI H4 (Biol. Pharm. Bull. 24; 214-217).

Abnormal levels of Apolipoprotein B48 receptor were also found to be indicative of the diagnosis of preterm delivery. On average, patients with term delivery exhibited a level of Apolipoprotein B48 receptor that was about 1.6 times higher as compared to patients with preterm delivery.

Levels of Apolipoprotein B48 receptor were measured using two dimensional differential gel electrophoresis (2D-DIGE) as described in the Examples below. For the routine determination of Apolipoprotein B48 receptor in clinical laboratories, an immunological method is preferred. Such methods are described in Example 10.

The gene encoding Apolipoprotein B48 receptor has been described in US 2003/0208060 and successful generation of specific antibodies has been disclosed therein. Moreover, mouse antibodies against human Apolipoprotein B48 receptor (clone 2D7) are commercially available from Abnova Corporation Neihu district, Taipei 114 Taiwan ROC. These antibodies can be used in a quantitative immunological assay for Apolipoprotein B48 receptor as further detailed in Example 10.

Abnormal levels of transferrin were also found to be indicative of the diagnosis of preterm delivery. On average, patients with term delivery exhibited a level of transferrin that was about 1.4 times higher as compared to patients with preterm delivery.

Levels of transferrin were measured using two dimensional differential gel electrophoresis (2D-DIGE) as described in the examples. For the routine determination of transferrin in the clinical laboratories, an immunological method is preferred. Such methods are described in Example 10. ELISA methods are particularly suitable. Example 12 details the measurement of transferrin in preterm delivery samples using a commercially available ELISA method. Immunological assays for human transferrin are commercially available. For example, an ELISA kit may be purchased at Bethyl Laboratories Montgomery, Tex. or from Agdia Inc., Alpco Diagnostics, American Research products Inc, Biomeda Corp., or Kamiya Biomedical Company.

In addition, abnormal levels of alpha-1B glycoprotein were found to be indicative of the diagnosis of preterm delivery. On average, patients with preterm delivery exhibited a level of alpha-1B glycoprotein that was about 1.4 times higher as compared to patients with term delivery.

Levels of alpha-1B glycoprotein were measured using two dimensional differential gel electrophoresis (2D-DIGE) as described in the following Examples. For the routine determination of alpha-1B glycoprotein in the clinical laboratories, an immunological method is preferred. Such methods are described in Example 10.

The gene encoding alpha-1B glycoprotein has been described in Strausberg R. L. et al, Proc. Natl. Acad. Sci. U.S.A. 99:16899-16903(2002), thus allowing the production of specific antibodies. Moreover, rabbit antibodies against human alpha-1B glycoprotein are commercially available from Genway Biotech Inc in San Diego and from Aviva Systems Biology San Diego.

Abnormal levels of antithrombin III were also found to be indicative of the diagnosis of preterm delivery. On average, patients with preterm delivery exhibited a level of antithrombin III that was about 1.7 times higher as compared to patients with term delivery.

Levels of antithrombin III were measured using two dimensional differential gel electrophoresis (2D-DIGE) as also described in the Examples. For the routine determination of antithrombin III in the clinical laboratories, an immunological method is preferred. Such methods are described in Example 10.

Mouse monoclonal antibodies against human antithrombin III are commercially available from AntibodyShop A/S in Gentofte, Denmark.

Further to the above, abnormal levels of apolipoprotein A-IV precursor were also found to be indicative of the diagnosis of preterm delivery. On average, patients with term delivery exhibited a level of apolipoprotein A-IV precursor that was about 1.8 times higher as compared to patients with preterm delivery.

Levels of apolipoprotein A-IV precursor were measured using two dimensional differential gel electrophoresis (2D-DIGE) as described in the examples. For the routine determination of apolipoprotein A-IV precursor in the clinical laboratories, an immunological method is preferred. Such methods are also described in Example 10.

Also, a quantitative ELISA for human apolipoprotein A-IV precursor has been described (Kondo et al., J. lipid Research 30, 939-944, 1989).

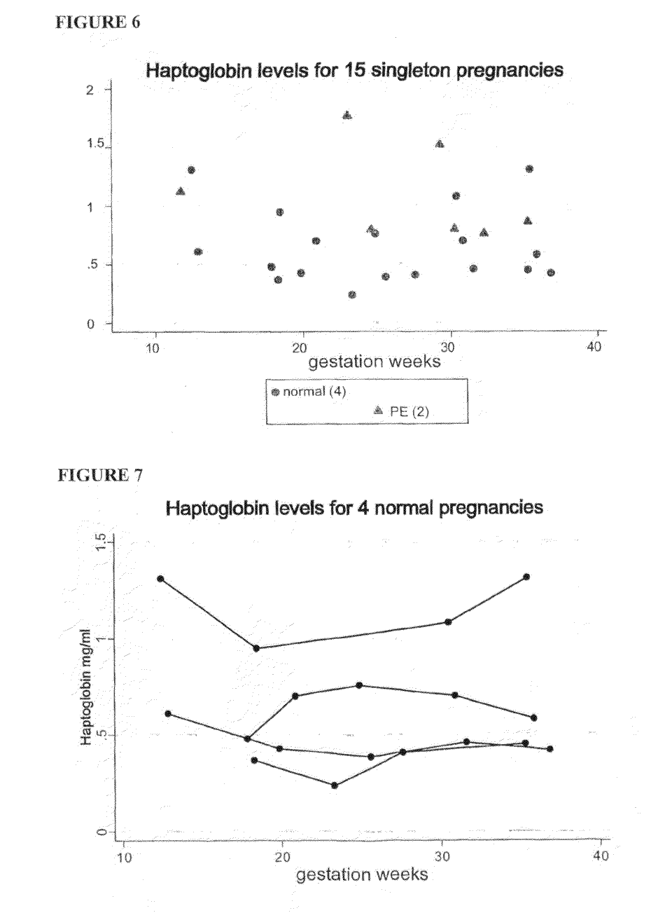

Abnormal levels of haptoglobin were found to be indicative for the diagnosis of pre-eclampsia. On average, patients with pre-eclampsia exhibited a level of haptoglobin that was higher as compared to patients with a normal pregnancy (FIG. 6).

Levels of haptoglobin were measured using an ELISA method as described in Example 13. For the routine determination of haptoglobin in clinical laboratories, an immunological method is preferred. Such methods are again described in Example 10.

Monoclonal and polyclonal antibodies against human haptoglobin are commercially available from AntibodyShop A/S, Grusbakken 8, DK-2820 Gentofte in Denmark and from Genway Biotech Inc in San Diego and from Aviva Systems Biology San Diego.

An ELISA for the sensitive quantitative determination of haptoglobin is commercially available from Gentaur, Av. de l' armee 68, B-1040 Brussels Belgium and from Genway Biotech Inc in San Diego.

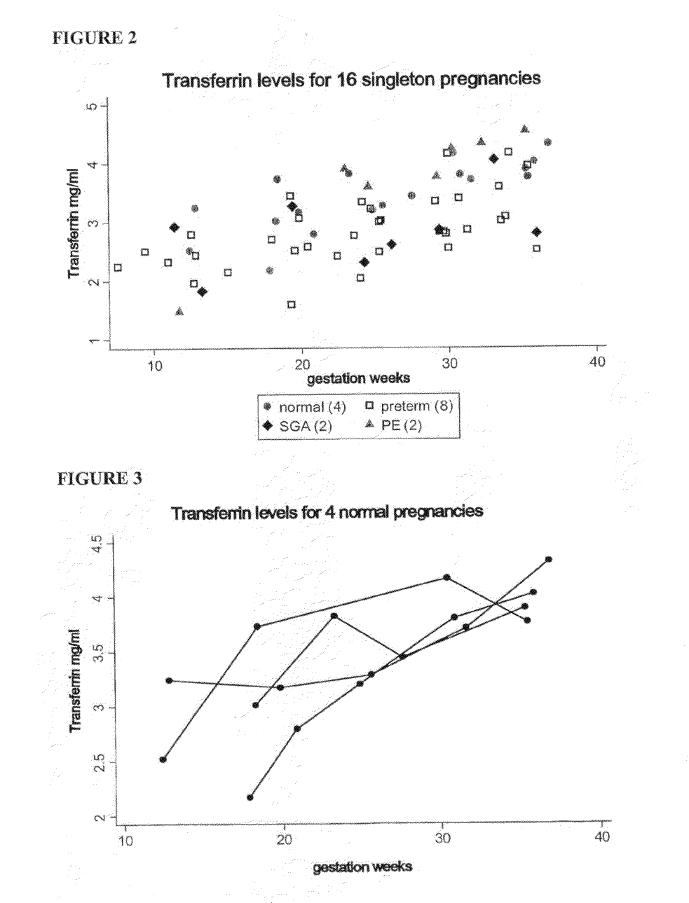

Abnormal levels of transferrin were also found to be indicative of the diagnosis of pre-eclampsia. On average, patients with pre-eclampsia exhibited a level of transferrin that was higher as compared to patients with normal delivery (FIG. 2).

Levels of transferrin were measured using an ELISA method as described in Example 12. For the routine determination of transferrin in the clinical laboratories, an immunological method is preferred. Such methods are described in Example 10. More preferred are immunological methods such as an ELISA method.

Immunological assays for human transferrin are commercially available. An ELISA kit may also be purchased at Bethyl Laboratories Montgomery, Tex. or from Agdia Inc., Alpco Diagnostics, American Research products Inc, Biomeda Corp., or Kamiya Biomedical Company.

Abnormal levels of alpha-1B glycoprotein were found to be indicative of the diagnosis of pre-eclampsia. On average, patients with pre-eclampsia exhibited a level of alpha-1B glycoprotein that was about 1.35 times higher as compared to patients with a normal pregnancy.

Abnormal levels of antithrombin III were also found to be indicative of the diagnosis of pre-eclampsia. On average, patients with pre-eclampsia exhibited a level of antithrombin III that was about 2 times higher as compared to patients with a normal pregnancy.

Abnormal levels of transthyretin were also found to be indicative of the diagnosis of pre-eclampsia. On average, patients with pre-eclampsia exhibited a level of transthyretin that was about 1.6 times lower as compared to patients with a normal pregnancy.

Levels of transthyretin were measured using two dimensional differential gel electrophoresis (2D-DIGE) as described in the examples. For the routine determination of transthyretin in the clinical laboratories, an immunological method is preferred. Such methods are described in Example 10.

The gene encoding transthyretin has been described by Mita et al., in Biochem. Biophys. Res. Commun. 124:558-564(1984), thus allowing the production of specific antibodies. Moreover, chicken antibodies against human transthyretin are commercially available from Genway Biotech Inc in San Diego and from Aviva Systems Biology San Diego.

An ELISA for the sensitive determination of transthyretin has been described by Vatassery et al., in Clin Chim. Acta, 1991, 197; 19-25.

Abnormal levels of haptoglobin were also found to be indicative for the diagnosis of pre-eclampsia. On average, patients with pre-eclampsia exhibited a higher level of haptoglobin as compared to patients with a normal pregnancy (FIG. 6).

Levels of haptoglobin were measured using an ELISA assay as described in example 13. For the routine determination of haptoglobin in the clinical laboratories, an immunological method is preferred. Such methods are described in Example 10.

Monoclonal and polyclonal antibodies against human haptoglobin are commercially available from AntibodyShop A/S, Grusbakken 8, DK-2820 Gentofte, Denmark, and from Genway Biotech Inc, San Diego, USA, and from Aviva Systems Biology, San Diego, USA.

An ELISA for the sensitive quantitative determination of haptoglobin is commercially available from Gentaur, Av. de l' armee 68, B-1040 Brussels Belgium and from Genway Biotech Inc, San Diego, USA.

Abnormal levels of transferrin were also found to be indicative of the diagnosis of IUGR. On average, patients with IUGR exhibited a level of transferrin that was lower as compared to patients with normal delivery (Example 12, FIG. 2).

Levels of transferrin were measured using an ELISA method as described in Example 12. For the routine determination of transferrin in the clinical laboratories, an immunological method is preferred. Such methods are described in Example 10. More preferred are immunological methods such as an ELISA method.

Immunological assays for human transferrin are commercially available. An ELISA kit may be purchased at Bethyl Laboratories Montgomery, Tex., USA, or from Agdia Inc., Alpco Diagnostics, American Research Products Inc, Biomeda Corp., or Kamiya Biomedical Company.

Abnormal levels of Inter-alpha trypsin Inhibitor heavy chain H4 (ITI H4) were found to be indicative for the diagnosis of IUGR. On average, patients with a normal pregnancy exhibited a level of ITI H4 that was about 2.3 times higher as compared to patients with IUGR.

Abnormal levels of antithrombin III were also found to be indicative for the diagnosis of IUGR. On average, patients with IUGR exhibited a level of antithrombin III that was about 1.8 times higher as compared to patients with a normal pregnancy.

Abnormal levels of haptoglobin were also found to be indicative for the diagnosis of IUGR. On average, patients with IUGR exhibited a level of haptoglobin that was about 1.9 times lower as compared to patients with a normal pregnancy.

Levels of haptoglobin were measured using two dimensional differential gel electrophoresis (2D-DIGE) as described in the examples. For the routine determination of haptoglobin in the clinical laboratories, an immunological method is preferred. Such methods are described in Example 10.

Monoclonal and polyclonal antibodies against human haptoglobin are commercially available from AntibodyShop A/S, Grusbakken 8, DK-2820 Gentofte, Denmark, and from Genway Biotech Inc, San Diego, USA, and from Aviva Systems Biology, San Diego, USA.

An ELISA for the sensitive quantitative determination of haptoglobin is commercially available from Gentaur, Av. de l' armee 68, B-1040 Brussels, Belgium, and from Genway Biotech Inc, San Diego, USA.

Abnormal levels of transthyretin were also found to be indicative for the diagnosis of IUGR. On average, patients with IUGR exhibited a level of transthyretin that was about 1.9 times lower as compared to patients with a normal pregnancy.

### Interpretation of Results

Previous biomarkers used for the determination of the risk of preterm delivery suffer from the drawback of a low sensitivity. The markers identified herein were found to be remarkably sensitive, at an excellent specificity. When the lowest measured value of the normal (term delivery) samples was taken as the cut-off value, the method employing anti-thrombin III was found to be 100% specific and 100% sensitive

(Table 3).

The values obtained for Inter-alpha trypsin Inhibitor heavy chain H4 varied remarkably within the normal (term delivery) group as well as within the preterm delivery group (Tables 1 and 2). At an average standardised abundance of 1.84, the standard deviation of 6 samples was 0.97. Due to this high variability, the sensitivity of this biomarker was the lowest among the group of 6 markers for preterm delivery disclosed herein. Nevertheless, Inter-alpha trypsin Inhibitor heavy chain H4 is considered a valuable and specific marker for preterm delivery since the average standardised abundance of the normal term delivery group is about twice the value of the preterm group. The sensitivity of this marker could be remarkably increased, even up to 100%, if a cut-off value was used such that the specificity of the assay was only slightly decreased, i.e. in the order of 80%.

Antithrombin III was found to be the most sensitive marker. It exhibited a specificity and sensitivity of 100% if the cut-off value was properly chosen. (Table 1).

Apolipoprotein B48 receptor and alpha-1B-glycoprotein were found to have a sensitivity of 80% when the cut-off values were chosen in such a way that the specificity of the assay would remain at 100% (Table 3). A sensitivity of 100% could he reached for both markers at 80% specificity at appropriately chosen cut-off values.

In combination assays wherein several of the markers presented herein were combined, an even more sensitive method could be obtained. This is completely in line with the aetiology of preterm delivery as it is usually considered to have multiple causes.

Almost any combination of two markers chosen from the six markers presented herein, resulted in a method with an increased sensitivity. Hence, the invention relates to a method for identifying a woman with an increased risk of premature delivery as described above, wherein at least two biomarkers were used, selected from the group consisting of transferrin, inter-alpha trypsin inhibitor heavy chain H4, apolipoprotein B48 receptor, alpha-IB-glycoprotein, antithrombin III and apolipoprotein A-IV precursor.

Particularly useful methods were those wherein at least two markers markers selected from the group consisting of transferrin, apolipoprotein B48 receptor, alpha-1B-glycoprotein, apolipoprotein A-IV precursor and antithrombin III were combined. Although it is evident from the results presented herein that any combination of biomarkers may increase the sensitivity of the method, in particular the following combinations provided complementary results: inter-alpha trypsin inhibitor heavy chain H4 plus transferrin, inter-alpha trypsin inhibitor heavy chain H4 plus apolipoprotein A-IV precursor, apolipoprotein B48 receptor plus alpha-1B-glycoprotein, apolipoprotein B48 receptor plus transferrin, apolipoprotein B48 receptor plus alpha-1B-glycoprotein, apolipoprotein B48 receptor plus antithrombin III, transferrin plus alpha-1B-glycoprotein, transferrin plus antithrombin III, alpha-1B-glycoprotein plus antithrombin III, alpha-1B-glycoprotein plus apolipoprotein A-IV precursor and antithrombin III plus apolipoprotein A-IV precursor.

Sensitivity of the method could even be further improved when three, four, five or even all six biomarkers were used in a method according to the invention. Even when the cut-off value of the assays was chosen at the average normal value plus or minus two times the standard deviation of the normal population, a combination assay could he created that was 100% specific and 100% specific. Even more importantly, it was found that on average two markers were positive for each of the six samples obtained from the preterm delivery patients (Table 2). In fact, the same results were obtained when using only four out of the six markers (Table 2). The only sample (#1) that was positive for only one marker (Alpha-1B-Glycoprotein) was not analysed against antithrombin III and Apolipoprotein A IV precursor because of a technical failure of the 2D gel analysis.

Current used biomarkers for the determination of the risk of pre-eclampsia also suffer from the drawback of a low sensitivity. The markers identified herein were found to be remarkably sensitive, at an excellent specificity.

When the lowest measured value of the normal (term delivery, non hypertensive disorder) samples was taken as the cut-off value, the method employing anti-thrombin III was found to be 100% specific and 100% sensitive (Table 6). Also, when the highest measured value of the normal (term delivery, non hypertensive disorder) samples was taken as the cut-off value, the method employing transthyretin was found to be 100% specific and 100% sensitive (Table 6).

The methods employing markers alpha-1B-glycoprotein and antithrombin III were found to be very robust; even when the mean value of the normal samples plus two times the standard deviation was taken as the cut off value, an excellent sensitivity was obtained of 75 and 100% respectively while the specificity remained at 100% (Table 5).

The values obtained for transthyretin varied remarkably within the normal (term delivery without hypertension) group as well as within the pre-eclampsia group (tables 4 and 5). At an average standardised abundance of 2,23, the standard deviation of 6 samples was 0.44. Due to this high variability, the negative predictive value of this biomarker was somewhat lower than the other two markers from the group of 3 markers disclosed herein. Nevertheless, transthyretin is considered a valuable and specific marker for pre-eclampsia since the average standardised abundance of the normal delivery group is about 1.6 times higher than the value of the pre-eclampsia group. The sensitivity of this marker was 100% at a specificity of 100% when an appropriate cut-off value was chosen.

Antithrombin III was found to be the most robust marker. It exhibited a specificity and sensitivity of 100%, even if the cut-off value was chosen at average normal value plus 2 times the standard deviation (Table 5).

An even more sensitive method for determination of the risk of pre-eclampsia could be obtained when several of the markers presented herein were combined, i.e. when combination assays were used. This is completely in line with the aetiology of pre-eclampsia as it is usually considered to have multiple causes.

Any combination of two markers chosen from the three markers presented herein, resulted in a method with an increased sensitivity. Hence, the invention relates to a method as described above, wherein at least two biomarkers were used, selected from the group consisting of alpha-1B-glycoprotein, antithrombin III and transthyretin.

Sensitivity of the method could even be further improved when all three biomarkers were used in a method according to the invention. Even when the cut-off value of the assays was chosen at the average normal value plus or minus two times the standard deviation of the normal population, a combination assay could be created that was 100% specific and 100% specific. Even more importantly, it was found that on average at least two markers were positive for each of the four samples obtained from the pre-eclampsia patients (Table 5).

A particularly useful result was obtained when markers alpha-1B-glycoprotein and antithrombin III were combined. Even if the cut-off value was chosen at average normal value plus 3 times the standard deviation, this method yielded a sensitivity and specificity of 75% and 100% respectively.

Current biomarkers for the determination of the risk of IUGR suffer from the drawback of a low sensitivity. The markers identified herein were found to be remarkably sensitive, at an excellent specificity.

When the lowest measured value of the normal (term delivery, non-hypertensive disorder, appropriate for gestational age) samples was taken as the cut-off value, the method employing anti-thrombin III was found to be 100% specific and 100% sensitive (Table 9).

The methods employing markers antithrombin III, haptoglobin and transthyretin were found to be very robust; even when the mean value of the normal samples plus or minus two times the standard deviation was taken as the cut off value, an excellent sensitivity was obtained of 100%, 50% and 50% respectively while the specificity remained at 100% (Table 8).

The values obtained for inter-alpha trypsin inhibitor heavy chain H4, varied remarkably within the normal (term delivery without hypertension) group as well as within the IUGR group (Tables 7 and 8). At an average standardised abundance of 1.84, the standard deviation of 6 normal samples was 0.97. Due to this high variability, the negative predictive value of this biomarker was somewhat lower than the other two markers from the group of 4 markers disclosed herein. Nevertheless, inter-alpha trypsin inhibitor heavy chain H4 is considered a valuable and specific marker for IUGR since the average standardised abundance of the normal delivery group is about 2.3 times higher than the value of the IUGR group. The sensitivity of this marker was 100% at a specificity of 75% when an appropriate cut-off value was chosen.

Antithrombin III was found to be the most robust marker. It exhibited a specificity and sensitivity of 100%, even if the cut-off value was chosen at average normal value plus 2 times the standard deviation (Table 8).

An even more sensitive method could be obtained when several of the markers presented herein were combined, i.e. when combination assays were used. This is completely in line with the aetiology of IUGR as it is usually considered to have multiple causes.

Any combination of two markers chosen from the three markers presented herein, resulted in a method with an increased sensitivity. Hence, the invention relates to a method as described above, wherein at least two biomarkers were used, selected from the group consisting of inter-alpha trypsin inhibitor heavy chain H4. antithrombin III, haptoglobin and transthyretin.

Sensitivity of the method could even be further improved when three or even all four biomarkers were used in a method according to the invention. Even when the cut-off value of the assays was chosen at the average normal value plus or minus two times the standard deviation of the normal population, a combination assay could be created that was 100% specific and 100% specific. Even more importantly, it was found that on average at least two markers were positive for each of the four samples obtained from the IUGR patients (table 8).

A particularly useful method was obtained when markers transthyretin and antithrombin III were combined. Even if the cut-off value was chosen at average normal value plus or minus almost 3 times the standard deviation, this method yielded a sensitivity and specificity of 100%.

TABLE 1
Standardised abundance of biomarkers indicative for preterm delivery in samples from 6 patients with term and 6 patients with
preterm delivery.

| | ITI H4 | | ALP B48 | | Transferrin | | alpha1B GP | | Antithrombin III | | ALP A IV precursor | |
|---|---|---|---|---|---|---|---|---|---|---|---|---|
| | Term | Preterm | Term | Preterm | Term | Preterm | Term | Preterm | Term | Preterm | Term | Preterm |
| | delivery | Delivery | delivery | Delivery | delivery | Delivery | delivery | Delivery | delivery | Delivery | delivery | Delivery |
| Sample # 1 | 0.66 | 1.35 | 1.30 | 1.23 | 0.76 | 0.66 | 1.04 | 1.14 | ND | ND | ND | ND |
| Sample # 2 | 1.56 | 0.82 | 1.51 | 0.75 | 0.55 | 0.54 | 0.96 | 1.46 | 0.82 | 1.39 | 0.47 | 0.58 |
| Sample # 3 | 1.59 | 1.13 | 1.38 | 1.01 | 1.16 | 0.77 | ND | ND | 0.47 | 1 | 1.39 | 0.53 |
| Sample # 4 | 2.16 | 0.3 | 1.60 | 1.03 | 0.89 | 0.72 | 0.91 | 1.43 | 0.62 | 1.08 | 1.13 | 0.66 |
| Sample # 5 | 3.55 | 1.01 | 1.13 | 0.75 | 0.77 | 0.51 | 0.8 | 1 | ND | ND | 1.15 | 0.22 |
| Sample # 6 | 1.51 | 0.69 | ND | ND | 1.12 | 0.56 | 0.92 | 1.15 | 0.6 | 1.15 | 0.79 | 0.39 |
| AVRG | 1.84 | 0.88 | 1.38 | 0.95 | 0.88 | 0.63 | 0.93 | 1.24 | 0.63 | 1.16 | 0.99 | 0.48 |
| STD | 0.97 | 0.37 | 0.18 | 0.21 | 0.23 | 0.11 | 0.09 | 0.20 | 0.14 | 0.17 | 0.36 | 0.17 |
| Cut-off | 0.66 | | 1.13 | | 0.55 | | 1.04 | | 0.82 | | 0.47 | |

ND = Not Determined.
Shaded samples represent positive results when the lowest or highest value of the normal range is taken as the cut-off value.

TABLE 2
Standardised abundance of biomarkers indicative for preterm delivery in samples from 6 patients with term and
6 patients with preterm delivery.

| | ITI H4 | | ALP B48 | | Transferrin | | alpha1B GP | | Antithrombin III | | ALP A IV precursor | |
|---|---|---|---|---|---|---|---|---|---|---|---|---|
| | Term | Preterm | Term | Preterm | Term | Preterm | Term | Preterm | Term | Preterm | Term | Preterm |
| | delivery | Delivery | delivery | Delivery | delivery | Delivery | delivery | Delivery | delivery | Delivery | delivery | Delivery |
| Sample # 1 | 0.66 | 1.35 | 1.30 | 1.23 | 0.76 | 0.66 | 1.04 | 1.14 | ND | ND | ND | ND |
| Sample # 2 | 1.56 | 0.82 | 1.51 | 0.75 | 0.55 | 0.54 | 0.96 | 1.46 | 0.82 | 1.39 | 0.47 | 0.58 |
| Sample # 3 | 1.59 | 1.13 | 1.38 | 1.01 | 1.16 | 0.77 | ND | ND | 0.47 | 1 | 1.39 | 0.53 |
| Sample # 4 | 2.16 | 0.3 | 1.60 | 1.03 | 0.89 | 0.72 | 0.91 | 1.43 | 0.62 | 1.08 | 1.13 | 0.66 |
| Sample # 5 | 3.55 | 1.01 | 1.13 | 0.75 | 0.77 | 0.51 | 0.8 | 1 | ND | ND | 1.15 | 0.22 |
| Sample # 6 | 1.51 | 0.69 | ND | ND | 1.12 | 0.56 | 0.92 | 1.15 | 0.6 | 1.15 | 0.79 | 0.39 |
| AVRG | 1.84 | 0.88 | 1.38 | 0.95 | 0.88 | 0.63 | 0.93 | 1.24 | 0.63 | 1.16 | 0.99 | 0.48 |
| STD | 0.97 | 0.37 | 0.18 | 0.21 | 0.23 | 0.11 | 0.09 | 0.20 | 0.14 | 0.17 | 0.36 | 0.17 |
| Cut-off | −0.09 | | 1.02 | | 0.41 | | 1.10 | | 0.92 | | 0.27 | |

ND = Not Determined.
Shaded samples represent positive results when the average normal value plus or minus 2 times the standard deviation is taken as the cut-off value.

TABLE 3
Sensitivity, specificity, positive predictive value and negative predictive value of
biomarkers for preterm delivery.

| | False | False | True | True | Specificity | Sensitivity | PPV | NPV |
|---|---|---|---|---|---|---|---|---|
| Biomarker | positive | negative | positive | negative | [%] | [%] | [%] | [%] |
| Inter-alpha trypsin inhibitor heavy | 0 | 5 | 1 | 6 | 100 | 17 | 100 | 55 |
| chain H4 | | | | | | | | |
| Apolipoprotein B48 receptor | 0 | 1 | 4 | 5 | 100 | 80 | 100 | 83 |
| Transferrin | 0 | 4 | 2 | 6 | 100 | 33 | 100 | 60 |
| Alpha-1B-glycoprotein | 0 | 1 | 4 | 5 | 100 | 80 | 100 | 83 |
| Antithrombin III | 0 | 0 | 4 | 4 | 100 | 100 | 100 | 100 |
| Apolipoprotein A-IV precursor | 0 | 4 | 2 | 6 | 100 | 33 | 100 | 60 |

Cut-off value = lowest/highest normal value.

TABLE 4
Standardised abundance of biomarkers indicative for pre-eclampsia in samples from 6 patients
with a normal pregnancy and 4 patients with pre-eclampsia.

| | alpha1B GP | | Antithrombin III | | Transthyretin | |
|---|---|---|---|---|---|---|
| | Normal pregnancy | Pre-eclampsia | Normal pregnancy | Pre-eclampsia | Normal pregnancy | Pre-eclampsia |
| Sample # 1 | 1.04 | 1.22 | ND | 1.19 | 2.75 | 1.07 |
| Sample # 2 | 0.96 | 1.00 | 0.82 | 1.00 | 1.98 | 1.77 |
| Sample # 3 | ND | 1.17 | 0.47 | 1.08 | 1.88 | 1.69 |
| Sample # 4 | 0.91 | 1.25 | 0.62 | 2.03 | 2.09 | 1.03 |
| Sample # 5 | 0.8 | ND | ND | ND | 2.81 | ND |
| Sample # 6 | 0.92 | ND | 0.6 | ND | 1.86 | ND |
| AVRG | 0.93 | 1.16 | 0.63 | 1.33 | 2.23 | 1.39 |
| STD | 0.09 | 0.16 | 0.14 | 0.48 | 0.44 | 0.39 |
| Cut-off | 1.04 | | 0.82 | | 1.86 | |

ND = Not Determined.
Shaded samples represent positive results when the lowest or highest value of the normal range is taken as the cut-off value.

TABLE 5
Standardised abundance of biomarkers indicative for pre-eclampsia in samples from 6 patients
with a normal pregnancy and 4 patients with pre-eclampsia.

| | alpha1B GP | | Antithrombin III | | Transthyretin | |
|---|---|---|---|---|---|---|
| | Normal pregnancy | Pre-eclampsia | Normal pregnancy | Pre-eclampsia | Normal pregnancy | Pre-eclampsia |
| Sample # 1 | 1.04 | 1.22 | ND | 1.19 | 2.75 | 1.07 |
| Sample # 2 | 0.96 | 1.00 | 0.82 | 1.00 | 1.98 | 1.77 |
| Sample # 3 | ND | 1.17 | 0.47 | 1.08 | 1.88 | 1.69 |
| Sample # 4 | 0.91 | 1.25 | 0.62 | 2.03 | 2.09 | 1.03 |
| Sample # 5 | 0.8 | ND | ND | ND | 2.81 | ND |
| Sample # 6 | 0.92 | ND | 0.6 | ND | 1.86 | ND |
| AVRG | 0.93 | 1.16 | 0.63 | 1.33 | 2.23 | 1.39 |
| STD | 0.09 | 0.16 | 0.14 | 0.48 | 0.44 | 0.39 |
| Cut-off | 1.10 | | 0.92 | | 1.36 | |

ND = Not Determined.
Shaded samples indicate positive results when the average normal value plus or minus 2 times the standard deviation is taken as the cut-off value.

TABLE 6
Sensitivity, specificity, positive predictive value and negative predictive value
of biomarkers for pre-eclampsia.

| | False | False | True | True | Specificity | Sensitivity | PPV | NPV |
|---|---|---|---|---|---|---|---|---|
| Biomarker | positive | negative | positive | negative | [%] | [%] | [%] | [%] |
| Alpha-1B-glycoprotein | 0 | 1 | 3 | 5 | 100 | 75 | 100 | 83 |
| Antithrombin III | 0 | 0 | 4 | 4 | 100 | 100 | 100 | 100 |
| Transthyretin | 0 | 0 | 4 | 6 | 100 | 100 | 100 | 100 |

Cut-off value = lowest/highest normal value.

TABLE 7
Standardised abundance of biomarkers indicative for IUGR in samples from 6 patients
with a normal pregnancy and 4 patients with IUGR.

| | | | Antithrombin | | | | | |
|---|---|---|---|---|---|---|---|---|
| | ITI H4 | | III | | Haptoglobin | | Transthyretin | |
| | Normal | | Normal | | Normal | | Normal | |
| | delivery | IUGR | delivery | IUGR | delivery | IUGR | delivery | IUGR |
| Sample # 1 | 3.55 | 0.75 | 0.82 | 1.02 | 1.60 | 0.84 | 2.81 | 1.53 |
| Sample # 2 | 2.16 | 1.10 | 0.62 | 1.45 | 1.31 | 0.74 | 2.75 | 1.93 |
| Sample # 3 | 1.59 | 0.74 | 0.60 | 0.98 | 1.23 | 0.46 | 2.09 | 0.67 |
| Sample # 4 | 1.56 | 0.56 | 0.47 | 0.94 | 1.01 | 0.34 | 1.98 | 0.61 |
| Sample # 5 | 1.51 | — | — | — | 0.94 | — | 1.88 | — |
| Sample # 6 | 0.66 | — | — | — | 0.84 | — | 1.86 | — |
| AVRG | 1.84 | 0.79 | 0.63 | 1.10 | 1.16 | 0.60 | 2.23 | 1.19 |
| STD | 0.97 | | 0.14 | | 0.28 | | 0.44 | |
| Cut-off | 0.66 | | 0.82 | | 0.84 | | 1.86 | |

ND = Not Determined.
Shaded samples represent positive results when the lowest or highest value of the normal range is taken as the cut-off value.

TABLE 8
Standardised abundance of biomarkers indicative for IUGR in samples from 6 patients
with a normal pregnancy and 4 patients with IUGR.

| | | | Antithrombin | | | | | |
|---|---|---|---|---|---|---|---|---|
| | ITI H4 | | III | | Haptoglobin | | Transthyretin | |
| | Normal | | Normal | | Normal | | Normal | |
| | delivery | IUGR | delivery | IUGR | delivery | IUGR | delivery | IUGR |
| Sample # 1 | 3.55 | 0.75 | 0.82 | 1.02 | 1.60 | 0.84 | 2.81 | 1.53 |
| Sample # 2 | 2.16 | 1.10 | 0.62 | 1.45 | 1.31 | 0.74 | 2.75 | 1.93 |
| Sample # 3 | 1.59 | 0.74 | 0.60 | 0.98 | 1.23 | 0.46 | 2.09 | 0.67 |
| Sample # 4 | 1.56 | 0.56 | 0.47 | 0.94 | 1.01 | 0.34 | 1.98 | 0.61 |
| Sample # 5 | 1.51 | — | — | — | 0.94 | — | 1.88 | — |
| Sample # 6 | 0.66 | — | — | — | 0.84 | — | 1.86 | — |
| AVRG | 1.84 | 0.79 | 0.63 | 1.10 | 1.16 | 0.60 | 2.23 | 1.19 |
| STD | 0.97 | | 0.14 | | 0.28 | | 0.44 | |
| Cut-off | −0.09 | | 0.92 | | 0.59 | | 1.36 | |

ND = Not Determined.
Shaded samples indicate positive results when the average normal value plus or minus 2 times the standard deviation is taken as the cut-off value.

TABLE 9
Sensitivity, specificity, positive predictive value and negative predictive
value of biomarkers for IUGR.

| | False | False | True | True | Specificity | Sensitivity | PPV | NPV |
|---|---|---|---|---|---|---|---|---|
| Biomarker | positive | negative | positive | negative | [%] | [%] | [%] | [%] |
| ITI H4 | 0 | 3 | 1 | 6 | 100 | 25 | 100 | 67 |
| Antithrombin III | 0 | 0 | 4 | 4 | 100 | 100 | 100 | 100 |
| Haptoglobin | 0 | 1 | 3 | 6 | 100 | 75 | 100 | 86 |
| Transthyretin | 0 | 1 | 3 | 6 | 100 | 75 | 100 | 86 |

Cut-off value = lowest/highest normal value.

The invention is further described below with reference to a number of non-limiting Examples.

### Example 1

### Plasma Samples

Samples were obtained prospectively from pregnant women who were followed until delivery and clinical conditions identified and monitored. Within a cohort of 500 pregnant women, 6 women were identified who experienced preterm birth (prior to 37 weeks), 6 women with pre-eclampsia and 6 women with IUGR. A group of normal pregnant women were also identified. Blood plasma samples from these four groups taken at 24 weeks of pregnancy were subjected to two dimensional difference gel electrophoresis (2D-DIGE) analysis. Using 2D-DIGE, the plasma protein profiles of 6 normal pregnant women were compared with plasma proteins profiles from the women who experienced preterm birth, pre-eclampsia or IUGR

### Example 2

### Depletion of HSA and IgG and Preparation of Blood Proteins

Human Serum Albumin (HSA) and gamma-immunoglobulin (IgG) account for up to 60% of total blood protein content. This is problematic for protein-based studies of blood because these proteins can mask and prevent the analysis of lower abundance proteins. Samples were depleted of HSA and IgG using the Albumin and IgG Removal Kit (GE Healthcare, Piscataway, N.J., USA) according to the manufacturer's protocol. In brief, extracted samples were incubated with an anti-HSA/anti-IgG resin for 30 mins at room temperature. Unbound proteins were separated from the absorption matrix using a mini-spin cartridge and centrifuged at 1000 rpm for 2 mins. To concentrate protein and remove excess salts, the HSA/IgG depleted samples were precipitated by adding 4 volumes of ice-cold acetone to each sample. The solution was then incubated for 3 hrs at −20° C. and then centrifuged at 13000 rpm for 15 mins at 4° C. Samples were resuspended in 50 μl of extraction buffer (7M urea, 2M thiourea, 30 mM Tris, 4% w/v CHAPS, pH 8.5). Protein concentration estimation was performed using the 2-D Quant

Kit (GE Healthcare).

### Example 3

### Fluorescent Cy-Dye Labelling of Samples

Prior to labelling, cyanine dyes Cy2, 3 and 5 (GE Healthcare) were reconstituted in anhydrous DMF. 50 μg protein from normal spontaneous labour and the preterm labour/IUGR/pre-eclampsia group were labelled with 400 pM of either Cy3 or

Cy5 dye on ice for 30 mins in the dark. Half of each sample population was labelled with Cy3 and the other with Cy5 to account for any dye binding bias. An internal pooled sample was created by mixing 25 μg of each sample and labelling as above with Cy2. Dye labelling reactions were stopped by adding 10 mM lysine and incubating for 10 mins on ice in the dark: Cy3 and Cy5-labelled samples were mixed with the Cy2-labelled pooled control and made up to a volume of 450 μl with rehydration buffer (7M urea, 2M thiourea, 4% w/v CHAPS, 2 mg/mL DTT and 1% v/v pH3-10 immobilised pH gradient (IPG) buffer).

### Example 4

### Two-Dimensional Gel Electrophoresis

In-gel rehydration of IPG strips (non linear, 013-10, 24 cm; GE Healthcare) was performed with the Cy-labelled samples overnight for 12 hrs at room temperature under mineral oil (Sigma, St. Louis, Mo., USA). Isoelectric focusing was performed using a Multiphor II unit (GE Healthcare) for a total of 58800 Vhrs using the following parameters; hold at 300V for 900 Vhrs; ramp and hold at 1000V for 3900 Vhrs; ramp and hold to 8000V for 13000 Vhrs; hold at 8000V for 41000 Vhrs. After focusing strips were equilibrated and reduced in equilibration buffer (30% v/v glycerol, 2% w/v SDS, 7M urea, trace bromophenol blue) with 0.5% DTT for 15 mins at room temperature and then in equilibration buffer containing 4.5% iodacetamide for 15 mins at room temperature. Equilibrated strips were applied on homogeneous 10% acrylamide gels (25.5×20.5 cm) cast in low-fluorescence plates using the Ettan™ DALTsix Electrophoresis Casting System (GE Healthcare). This enabled the production of six simultaneously-cast gels using the same batch of acrylamide gel stock solution for each gel. Second-dimensional electrophoretic separation was carried out at 2.5 W/gel constant power for 30 mins then 100 W (total) constant power using a peltier-cooled Ettan DALT II electrophoresis unit (GE Healthcare) until the bromophenol dye front reached the bottom of the gel.

### Example 5

### Imaging and Analysis of 2D-DIGE Gels

Fluorescence image acquisition was performed using the Typhoon 9400 Variable Mode Imager (GE Healthcare). Each gel was scanned at the following excitation/emission wavelengths; 480/530 nm (Cy2), 520/590 nm (Cy3) and 620/680 nm (Cy5). Gel analysis was conducted using the DeCyder V5.0 Biological Variation software module (GE Healthcare). This enabled spot matching between gels and also to quantify and standardise protein spot data. Spot matching was validated by manual inspection of gel spots. Statistically significant protein abundance changes were identified via Student's T-test. Proteins that were present in either all gels or at least in the spontaneous normal labour group with a p-value of p<0.05 or less were chosen as proteins for identification.

### Example 6

### Utilisation of the Internal Standard

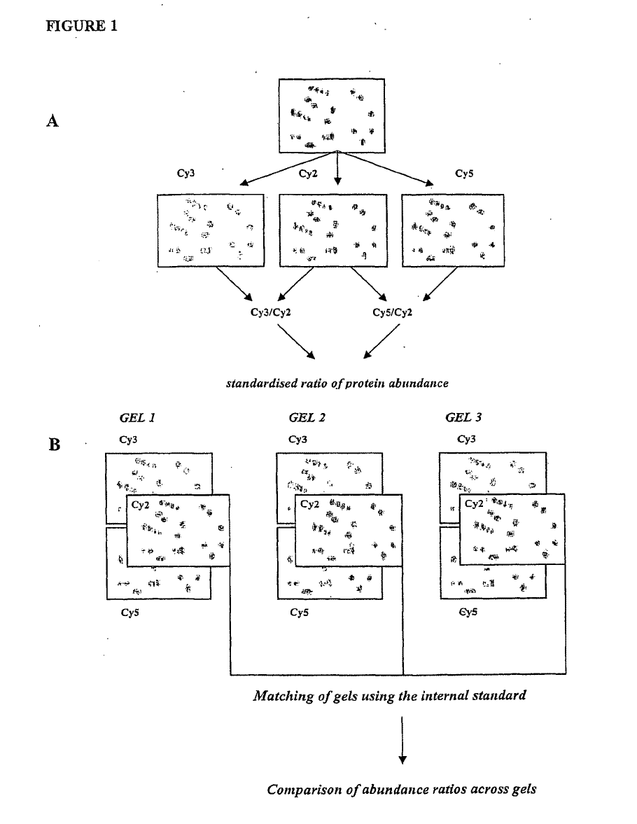

The primary advantage of 2D-DIGE over other proteomic techniques is the inclusion of an internal standard control. This allows to compensate for variation within gels and across gels in an experimental series. The internal control was created by pooling equal quantities of all samples to be assessed in each experiment. The pool was labelled with one of the Cy fluorophores (Cy2) and applied to every gel in the experiment containing Cy3 and Cy5 labelled samples. This means that every individual protein spot on the gel was be represented by the Cy2-labelled internal control. Therefore each protein spot could be standardised to its own control spot as well as across all gels in the experiment (FIG. 1). In this way gel-to-gel variation was eliminated. This also allowed for the discrimination between system variation and true biological variation.

### Example 7

### Protein Sequencing

Two sequencing gels were prepared by performing 2D-PAGE on 500 μg and 1000 μg of pooled myometrium proteins. The gels were fixed in 50% methanol, 7% acetic acid for 30 mins at room temperature and then stained with SYPRO Ruby (Invitrogen, California, USA) overnight also at room temperature. The gels were then washed in 10% methanol, 7% acetic acid and briefly rinsed in distilled water before being visualised using a UV illuminator. Proteins of interest were excised from the gels and destained in 50 mM ammonium bicarbonate in 50% v/v methanol for 3 washes of 40 mins each. Gel plugs were then dried at 37° C. for 2 hrs and then incubated in 40 ng/μl trypsin (Promega, Madison, Wis., USA) in 20 mM ammonium bicarbonate at 37° C. for 3 hours. Protein sequencing was performed via matrix-assisted laser desorption time-of-flight mass spectrometry (MALDI-ToF) using the Ettan MALDI-ToF Pro (GE Healthcare). Approximately 10 tryptic peptide was mixed with 1 μl a-cyano-4-hydroxycinnamic acid matrix (5 mg/ml in 50% acetonitrile, 0.1% trifluroacetic acid) and 1 μl of this mixture was spotted in duplicate onto the Maldi target tray. Peptide mass fingerprinting was performed alongside peptide standards (company) in reflectron mode. The external standard was use to calibrate the mass spectrometer prior to acquisition of sample MS data acquisition. Sequence data was used to interrogate the Swiss-Prot and NCBInr databases. As a further control measure, the isoelectric point and molecular weight of positively identified proteins were checked against the region of the pick gel they were excised from.

The following procedures are constructively reduced to practice and therefore described in the present tense, as opposed to the previous examples which were carried out in the laboratory and which are therefore set forth in the past tense.

### Example 8

### Purification of Polypeptide Biomarkers

The polypeptide biomarkers detectable in methods embodied by the invention can be produced by first cloning the corresponding polynucleotides into a mammalian expression vector using established protocols that are familiar to those in skill of the art. In brief, the polynucleotides are amplified and isolated via agarose gel electrophoresis and gel excision. The polynucleotides are subsequently cloned into appropriate vectors to facilitate in-frame cloning. Proteins may for instance be expressed with a polyhistidine metal-binding tag. The vectors incorporating the polynucleotides can then be transfected into a mammalian cell line like COS-7 for high-level expression of the corresponding polypeptides.

The primary nucleotide sequences of the biomarkers detactable in methods embodied by the invention may be obtained from GenBank, National Centre for Biotechnology Information, National Library of Medicine, Bethesda, Md., United States (Tables 10-12).

The biomarkers can be produced by the transfected mammalian cell line and then be purified according to established protocols that are familiar to those of skill in the art, for example using the Ni-NTA Magnetic Agarose Beads protein purification protocol, Qiagen, Valencia, Calif. In brief, assays utilizing Ni-NTA Magnetic Beads involve capture of the His-tagged protein-from a cell lysate or a purified-protein solution-followed by washing, binding of interaction partners, further washing, and finally elution of the interacting partner from the still immobilized His-tagged protein or elution of the interacting-partner-His-tagged-protein complex. Between each step, the beads are collected by attracting them to the side of the vessel, after placing near a magnet for 30-60 seconds. Purification procedures may even use crude cell extracts for binding of His-tagged protein.

The resulting purified polypeptides are then quantified, lyophilized and stored at 4 ° C. or below, and may be used as standards in diagnostic kits or for the generation of antibodies.

### Example 9

### Production of Specific Antibodies

Polyclonal antibodies are produced by DNA vaccination or by injection of peptide antigens into rabbits or other hosts. An animal, such as a rabbit, is immunized with a peptide advantageously conjugated to a carrier protein, such as BSA (bovine serum albumin) or KLH (keyhole limpet hemocyanin). The rabbit is initially immunized with conjugated peptide in complete Freund's adjuvant. followed by a booster shot every two weeks with injections of conjugated peptide in incomplete Freund's adjuvant. Antibodies specific for the polypeptide of interest are affinity purified from rabbit serum using peptide fragments of the polypeptides, for instance coupled to Affi-Gel 10 (Bio-Rad), and stored in phosphate-buffered saline with 0.1% sodium azide. To determine that polyclonal antibodies are specific for the relevant polypeptide, an expression vector encoding the polypeptide is introduced into mammalian cells. Western blot analysis of protein extracts of non-transfected cells and the cells expressing the polypeptide is performed using the polyclonal antibody sample as the primary antibody and a horseradish peroxidase-labeled anti-rabbit antibody as the secondary antibody. Detection of a band in the cells containing the polypeptide of interest and lack thereof in the control cells indicates that the polyclonal antibodies are specific for the polypeptide in question.

Monoclonal antibodies are produced by injecting mice with a peptide derived from the biomarker, with or without adjuvant. Subsequently, the mouse is boosted every 2 weeks until an appropriate immune response has been identified (typically 1-6 months), at which point the spleen is removed. The spleen is minced to release splenocytes, which are fused (in the presence of polyethylene glycol) with murine myeloma cells. The resulting cells (hybridomas) are grown in culture and selected for antibody production by clonal selection. The antibodies are secreted into the culture supernatant, facilitating the screening process, such as screening by an enzyme-linked immunosorbent assay (ELISA). Alternatively, humanized monoclonal antibodies are produced either by engineering a chimeric murine/human monoclonal antibody in which the murine-specific antibody regions are replaced by the human counterparts and produced in mammalian cells, or by using transgenic “knock out” mice in which the native antibody genes have been replaced by human antibody genes and immunizing the transgenic mice as described above. Suitable antibodies may also be commercially obtained as described above.

### Example 10

### Diagnostic Kit for Detection of Biomarkers

A diagnostic kit can be made in order to determine the levels of the biomarker polypeptide(s) that are present in samples obtained from a pregnant patient. Such samples may convenieintly be from blood, blood plasma, blood serum, amniotic fluid, saliva or urine.

A diagnostic kit may comprise some or all of the following components: 1) one or more standards comprised of one or more of the biomarker(s) prepared as described in Example 8; 2) an antibody or a plurality of antibodies that are specific for the biomarker(s) that are to be assayed for using the kit, prepared as described in Example 9; 3) written instructions; 4) diluents for samples and the standards; 5) a wash buffer; 6) color reagents; 7) stop solution; and 8) an antibody carrier such as polystyrene beads or a lateral flow device or a microplate with bound antibody.

An example of such a kit is a quantificative ELISA (enzyme-linked immunosorbent assay) that determines the concentration or concentrations of the biomarker or biomarker(s) in accordance with methods embodied by the invention. The principle of the assay is to use the quantitative sandwich enzyme immunoassay technique wherein a monoclonal or polyclonal antibody selective for a biomarker is pre-coated onto a carrier such as a microplate into its wells. The standards and sample are then pipetted into the wells and any of the biomarker that is present is bound to this immobilized antibody. Next, the wells are washed with washing buffer, and an enzyme-linked monoclonal or polyclonal antibody that is specific for the biomarker is added to the wells. Washing is again performed, then a substrate solution is added to the wells. Color subsequently develops in proportion to the amount of polypeptide of the invention that is bound in the first step. The color development is stopped using a stop solution, and the intensity of the color is measured by a microplate reader.

Alternatively, a lateral flow assay may be developed. Such lateral flow assays have the potential to be a cost-effective, fast, simple, and sensitive method, for instance for on-site screening assays. The principle is well known to the skilled person and consist essentially of a carrier that allows a lateral flow to occur wherein either the sample or the detection reagent is displaced form one location on the carrier to another. There are many formats of lateral flow assays suitable for use in a method embodied by the invention, and the skilled person will readily know how to select and optimize a particular format.

### Example 11

### Diagnostic Chip for Determination of Levels of Biomarkers

The levels of biomarkers in biological samples as described herein can also be determined through the use of ProteinChip technology (Wright Jr., et al. Expert Review of Molecular Diagnositcs 2002, Vol. 2 pp. 549-63, the entire contents of which are hereby incorporated herein by reference). This process includes the additon of a few microliters of sample onto the ProteinChip surface, which has both chemical (e.g., anionic, catioinic, hydrophobic, hydrophilic, metal) and biochemical (antibody, receptor, DNA, enzyme probes attached to its surface. Subsequently, binding occurs between the polypeptides of the invention and their respective probes, and the chip is washed at least three times in order to elute unbound protein, lipids, salts, and other substances. Next, an energy-absorbent molecule (EAM) such as sinapinic acid is added to the chip, and the chip is placed into a ProteinChip Reader which measures the molecular mass of the bound polypeptides. Peaks are subsequently produced from the ProteinChip Reader that are used to quantify the amount of the target polypeptides in the sample.

### Example 12

### Confirmation of Diagnostic Potential of Transferrin

An AssayMax Human Transferrin ELISA Kit was purchased from AssayPro (Catalogue Number ET2105-01). This assay uses an anti-human transferrin antibody coated on a 96 wells ELISA plate. Transferrin from standards and patient plasma samples competes with biotinylated transferrin from the kit. A Streptavidin-peroxidase conjugate was finally used for detection. Results for each pregnancy were fitted to a linear equation using regression:

transferrin=tr_{—}a+tr_{—}b*t where t is gestation days

If the linear fit is acceptable, then levels can be calculated for any week in the range and the slope (or rate of change) of Transferrin may also prove useful to predict PTD, PE and SGA pregnancies. The combined residuals were normally distributed and the R² squared (goodness of fit) statistics were 0.6 (mean), 0.01 (min), 0.98 (max); several trajectories fitted very well and several very poorly. This is shown in FIGS. 2 and 3.

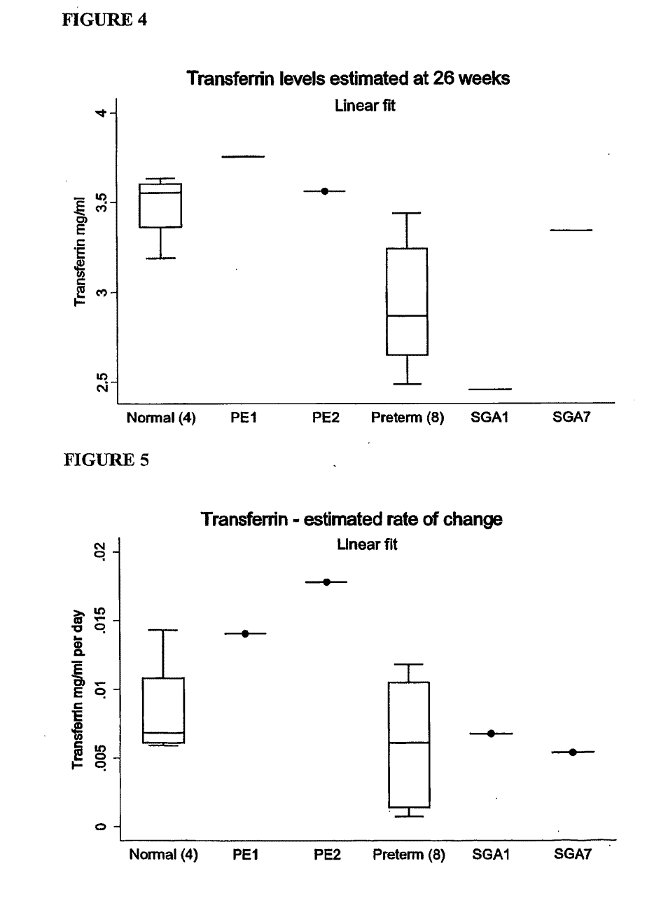

The box and whiskers plots shown in FIGS. 4 and 5 show the Transferrin levels estimated at 26 weeks and the rate of change. 26 weeks was chosen as it has proved useful for predicting PTD from CRH, hCG and Cortisol levels. The two PE and two SGA pregnancies are shown separately.

### Example 13

### Confirmation of Diagnostic Potential of Haptoglobin

An AssayMax Human Haptoglobin ELISA Kit from AssayPro (Cat. No. EH1003-1) was purchased for these studies. This assay uses an anti-human haptoglobin antibody coated on a 96 wells ELISA plate. Haptoglobin from standards and patient plasma samples competes with biotinylated haptoglobin from the kit. A Streptavidin-peroxidase conjugate was used for detection.

Pre-eclampsia pregnancies appear to have higher than normal Haptoglobin levels (FIG. 6)

TABLE 10
List of high molecular weight proteins identified as biomarkers of preterm delivery.

| | | | MW of | | | |
|---|---|---|---|---|---|---|
| | | | unprocessed | | | |
| | Swiss Prot accession # | | precurser | | Peptides | % sequence |
| Protein ID | and entry name | Synonyms | (KDa) | pI | Sequenced | coverage |
| Inter-alpha-trypsin | Q14624, | ITI heavy chain H4, Inter-alpha-inhibitor | 103.358 | 6.5 | 7 | 8.8 |
| heavy chain H4 | ITIH4_HUMAN | heavy chain 4, Inter-alpha-trypsin | | | | |
| | | inhibitor family, heavy chain-related | | | | |
| | | protein, IHRP, Plasma kallikrein sensitive | | | | |
| | | glycoprotein 120, PK-120, GP120 | | | | |
| Apolipoprotein | Q9NPJ9, | | 114.835 | 4.4 | 6 | 8.7 |
| B48 receptor | Q9NPJ9_HUMAN | | | | | |
| Transferrin | P02787 | serotransferrin, siderophilin, beta-1-metal- | 77.05 | 6.9 | 21 | 26.1 |
| | TRFE_HUMAN | binding globulin | | | | |
| Alpha-1B- | P04217 | | 54.273 | 5.6 | 5 | 12.3 |
| glycoprotein | AIBG_HUMAN | | | | | |
| Antithrombin III | P01008 | ATIII | 52.602 | 6.3 | 6 | 11.9 |
| | ANT3_HUMAN | | | | | |
| Apolipoprotein-A- | P06727 | Apo-AIV, ApoA-IV | 45.399 | 5.3 | 14 | 36.6 |
| IV precursor | APOA4_HUMAN | | | | | |

TABLE 11
List of high molecular weight proteins identified as biomarkers of pre-eclampsia.

| | | | | | | % |
|---|---|---|---|---|---|---|
| | | | MW of unprocessed | | Peptides | sequence |
| Protein ID | Swiss Prot accession # and entry name | Synonyms | precurser (KDa) | pI | Sequenced | coverage |
| Alpha-1B- | P04217 AIBG_HUMAN | | 54.273 | 5.6 | 5 | 12.3 |
| glycoprotein | | | | | | |
| Antithrombin | P01008 ANT3_HUMAN | ATIII | 52.602 | 6.3 | 6 | 11.9 |
| III | | | | | | |
| Transthyretin | Q549C7 Q549C7_HUMAN | Prealbumin, TBPA, | 15.887 | 5.3 | 6 | 74.4 |
| | | TTR, ATTR | | | | |
| Transferrin | P02787 TRFE_HUMAN | serotransferrin, | 77.05 | 6.9 | 21 | 26.1 |
| | | siderophilin, beta-1- | | | | |
| | | metal-binding | | | | |
| | | globulin | | | | |
| Haptoglobin | P00738 HPT_HUMAN | | 45.399 | 6.1 | 5 | 10.4 |

TABLE 12
List of high molecular weight proteins identified as biomarkers of IUGR.

| | | | MW of | | | |
|---|---|---|---|---|---|---|
| | | | unprocessed | | | % |
| | Swiss Prot accession # and | | precurser | | Peptides | sequence |
| Protein ID | entry name | Synonyms | (KDa) | pI | Sequenced | coverage |
| Inter-alpha- | Q14624, ITIH4_HUMAN | ITI heavy chain H4, Inter-alpha-inhibitor heavy | 103.358 | 6.5 | 7 | 8.8 |
| trypsin heavy | | chain 4, Inter-alpha-trypsin inhibitor family, | | | | |
| chain H4 | | heavy chain-related protein, IHRP, Plasma | | | | |
| | | kallikrein sensitive glycoprotein 120, PK-120, | | | | |
| | | GP120 | | | | |
| Antithrombin | P01008 ANT3_HUMAN | ATIII | 52.602 | 6.3 | 6 | 11.9 |
| III | | | | | | |
| Haptoglobin | P00738 HPT_HUMAN | | 45.399 | 6.1 | 5 | 10.4 |
| Transthyretin | Q549C7 | Prealbumin. TBPA. TTR. ATTR | 15.887 | 5.3 | 6 | 74.4 |
| | Q549C7_HUMAN | | | | | |
| Transferrin | P02787 TRFE_HUMAN | serotransferrin, siderophilin, beta-1-metal- | 77.05 | 6.9 | 21 | 26.1 |
| | | binding globulin | | | | |

1. A method for identifying a woman with an increased risk of premature delivery, comprising the steps of:
(a) obtaining a biological sample from a pregnant patient after week 12 and prior to week 37 of pregnancy;
(b) determining the level of at least one biomarker in the sample, the biomarker being selected from the group consisting of transferrin, inter-alpha trypsin inhibitor heavy chain H4, apolipoprotein B48 receptor, alpha-1B-glycoprotein, antithrombin III and apolipoprotein A-IV precursor;
(c) comparing the level of the at least one biomarker with a level characteristic of normal pregnancy; and
(d) determining whether the patient has an increased risk of premature delivery from the comparison.

2. A method according to claim 1 wherein an abnormal level of the biomarker is indicative of an increased risk of premature delivery, and the abnormal level is either a decreased level of transferrin, inter-alpha trypsin inhibitor heavy chain H4, apolipoprotein B48 receptor or apolipoprotein A-IV precursor or an increased level of alpha-1B-glycoprotein or antithrombin 111.

3. A method according to claim 1 or 2 which is an immunological method.

4. A method according to claim 3 which is a radioimmunoassay, ELISA or a lateral flow assay.

5. A method according to any one of claims 1 to 4 wherein the at least one biomarker is selected from the group consisting of transferrin, apolipoprotein B48 receptor, alpha-1B-glycoprotein and antithrombin HI.

6. A method according to claims any one of claims 1 to 5 wherein the biological sample is blood, plasma or serum.

7. A method for preventing a preterm delivery, comprising the steps of identifying a woman with an increased risk of premature delivery as defined in any one of claims 1 to 6, and treating the woman with a tocolytic agent.

8. A method for ameliorating the effects of a preterm delivery for the newborn, comprising the steps of identifying a woman with an increased risk of premature delivery as defined in any one of claims 1 to 6, and treating the woman with a steroid or another effective medication in order to promote foetal lung maturation or delay delivery.

9. A method for determining whether a given pregnancy is likely to proceed to full term, comprising the steps of:
(a) obtaining a biological sample from a pregnant patient after week 12 and prior to week 37 of pregnancy;
(b) determining the level of at least one biomarker in the sample, the biomarker being selected from the group consisting of transferrin, inter-alpha trypsin inhibitor heavy chain H4, apolipoprotein B48 receptor, transferrin, alpha-1B-glycoprotein, antithrombin III and apolipoprotein A-IV precursor;
(c) comparing the level of the at least one biomarker with a level characteristic of normal pregnancy; and
(d) determining whether the pregnancy of the patient is likely to proceed to term from the comparison.

10. A method for identifying a woman with an increased risk of pre-eclampsia or developing a hytertensive disorder associated with pregnancy, comprising the steps of:
(a) obtaining a biological sample from a pregnant patient after week 12 and prior to week 37 of pregnancy;
(b) determining the level of at least one biomarker in the sample, the biomarker being selected from the group consisting of transferrin, alpha-1B-glycoprotein, antithrombin III, haptoglobin and transthyretin;
(c) comparing the level of the at least one biomarker with a level characteristic of normal pregnancy: and
(d) determining whether the patient has an increased risk of pre-eclampsia or developing a hypertensive disorder from the comparison.

11. A method according to claim 10 wherein an abnormal level of the biomarker is indicative of an increased risk of pre-eclampsia or developing a hypertensive disorder, and the abnormal level is either a decreased level of transthyretin or an increased level of transferrin, haptoglobin, alpha-1B-glycoprotein or antithrombin III.

12. A method according to claim 10 or 11 which is an immunological method.

13. A method according to claim 12 which is a radioimmunoassay, ELISA or a lateral flow assay.

14. A method according to claims any one of claims 10 to 13 wherein the at least one biomarker is selected from the group consisting of alpha-1B-glycoprotein and antithrombin III.

15. A method according to any one of claims 10 to 14 wherein the biological sample is blood, plasma, or serum.

16. A method according to any one of claims 10 to 15 for identifying a woman with an increased risk of pre-eclampsia.

17. A method according to any one of claims 10 to 15 for identifying a woman with an increased risk of developing a hypertensive disorder.

18. A method for ameliorating the effects of a preterm delivery for the newborn, comprising the steps of identifying a woman with an increased risk of pre-eclampsia as defined in any one of claims 10 to 17, and treating the woman with a steroid or another effective medication in order to promote foetal lung maturation.

19. A method for screening or pre-screening pregnant women to identify those pregnant women with a low risk of developing a hytertensive disorder associated with pregnancy, comprising the steps of:
(a) obtaining a biological sample from a pregnant patient after week 12 and prior to week 37 of pregnancy;
(b) determining the level of at least one biomarker in the sample, the biomarker being selected from the group consisting of transferrin, alpha-1B-glycoprotein, antithrombin III, haptoglobin and transthyretin;
(c) comparing the level of the at least one biomarker with a level characteristic of normal pregnancy; and
(d) determining whether the patient is at low risk of developing a hypertensive disorder from the comparison.

20. A method for identifying a woman with an increased risk of IUGR, comprising the steps of:
(a) obtaining a biological sample from a pregnant patient after week 12 and prior to week 37 of pregnancy;
(b) determining the level of at least one biomarker in the sample, the biomarker being selected from the group consisting of transferrin, inter-alpha trypsin inhibitor heavy chain H4, antithrombin III, haptoglobin and transthyretin;
(c) comparing the level of the at least one biomarker with a level characteristic of normal pregnancy; and
(d) determining whether the patient has an increased risk of IUGR from the comparison.

21. A method according to claim 20 wherein an abnormal level of the biomarker is indicative of increased risk of IUGR, and the abnormal level is either a decreased level of transferrin, inter-alpha trypsin inhibitor heavy chain H4, haptoglobin and transthyretin or a decreased level of antithrombin

22. A method according to claim 20 or 21 which is an immunological method.

23. A method according to claim 22 which is a radioimmunoassay, ELISA or a lateral flow assay.

24. A method according to any one of claims 20 to 23 wherein the at least one biomarker is antithrombin III.

25. A method according to any one of claims 20 to 24 wherein the biological sample is blood, plasma or serum.

26. A method for ameliorating the effects of a preterm delivery for the newborn comprising the steps of identifying a woman with an increased risk of IUGR as defined in any one of claims 20 to 25, and treating the woman with a steroid or another effective medication in order to promote foetal lung maturation.

27. A method for screening or pre-screening pregnant women to identify those pregnant women with a low risk of developing IUGR, comprising the steps of:
(a) obtaining a biological sample from a pregnant patient after week 12 and prior to week 37 of pregnancy;
(b) determining the level of at least one biomarker in the sample, the biomarker being selected from the group consisting of transferrin, inter-alpha trypsin inhibitor heavy chain H4, antithrombin III, haptoglobin and transthyretin;
(c) comparing the level of the at least one biomarker with a level characteristic of a normal pregnancy; and
(d) determining whether the patient has a low risk of developing IGUR from the comparison.
